SEQUENCE LISTING (43 sequences)

SEQ ID NO: 1 - DNA, 30 bp, synthetic construct
  source 1..30: mol_type = other DNA
aactgaattcttgacaattaatcatccggc

SEQ ID NO: 2 - DNA, 32 bp, synthetic construct
  source 1..32: mol_type = other DNA
ctggagatctagttaatttctcctctttaatg

SEQ ID NO: 3 - DNA, 27 bp, synthetic construct
  source 1..27: mol_type = other DNA
attatctagaatgagaaaccctgatac

SEQ ID NO: 4 - DNA, 28 bp, synthetic construct
  source 1..28: mol_type = other DNA
gcgtactagtctagaaattaagttcggc

SEQ ID NO: 5 - DNA, 28 bp, synthetic construct
  source 1..28: mol_type = other DNA
cggctctagaatgttttcaaaagaagtc

SEQ ID NO: 6 - DNA, 29 bp, synthetic construct
  source 1..29: mol_type = other DNA
gcgtactagtttacaaaggaccggaaatg

SEQ ID NO: 7 - DNA, 32 bp, synthetic construct
  source 1..32: mol_type = other DNA
cggctctagaatgttagtaaaaatccttaaat

SEQ ID NO: 8 - DNA, 27 bp, synthetic construct
  source 1..27: mol_type = other DNA
cgtgactagtctaagcccaccagggat

SEQ ID NO: 9 - DNA, 29 bp, synthetic construct
  source 1..29: mol_type = other DNA
cgggtctagaatgagtattatcaaaaagc

SEQ ID NO: 10 - DNA, 27 bp, synthetic construct
  source 1..27: mol_type = other DNA
cgtgactagtttagaacaagcccaagg

SEQ ID NO: 11 - DNA, 43 bp, synthetic construct
  source 1..43: mol_type = other DNA
attttctagaaggagatataatatggtgagcaagggcgaggag

SEQ ID NO: 12 - DNA, 31 bp, synthetic construct
  source 1..31: mol_type = other DNA
actgactagtttattacttgtacagctcgtc

SEQ ID NO: 13 - DNA, 31 bp, synthetic construct; identical to SEQ ID NO: 12
  source 1..31: mol_type = other DNA

SEQ ID NO: 14 - DNA, 40 bp, synthetic construct
  source 1..40: mol_type = other DNA
ttcacacaggagatataatatgagaaaccctgatactttg

SEQ ID NO: 15 - DNA, 23 bp, synthetic construct
  source 1..23: mol_type = other DNA
attatatctcctgtgtgaaattg

SEQ ID NO: 16 - DNA, 42 bp, synthetic construct
  source 1..42: mol_type = other DNA
cttcttgagatcctttttttgaattcttgacaattaatcatc

SEQ ID NO: 17 - DNA, 43 bp, synthetic construct
  source 1..43: mol_type = other DNA
caatttcacacaggagatataatatgttttcaaaagaagtcac

SEQ ID NO: 18 - DNA, 43 bp, synthetic construct; identical to SEQ ID NO: 17
  source 1..43: mol_type = other DNA

SEQ ID NO: 19 - DNA, 44 bp, synthetic construct
  source 1..44: mol_type = other DNA
caatttcacacaggagatataatatgttagtaaaaatccttaaa

SEQ ID NO: 20 - DNA, 44 bp, synthetic construct; identical to SEQ ID NO: 19
  source 1..44: mol_type = other DNA

SEQ ID NO: 21 - DNA, 41 bp, synthetic construct
  source 1..41: mol_type = other DNA
atttcacacaggagatataatatgagtattatcaaaaagcc

SEQ ID NO: 22 - DNA, 39 bp, synthetic construct
  source 1..39: mol_type = other DNA
attatatctccttctagattagaacaagcccaaggttag

SEQ ID NO: 23 - DNA, 31 bp, synthetic construct
  source 1..31: mol_type = other DNA
ttcatctagactagaaattaagttcggcagc

SEQ ID NO: 24 - DNA, 26 bp, synthetic construct
  source 1..26: mol_type = other DNA
gaacgaattcttgacaattaatcatc

SEQ ID NO: 25 - DNA, 31 bp, synthetic construct
  source 1..31: mol_type = other DNA
attctctagattagaacaagcccaaggttag

SEQ ID NO: 26 - DNA, 24 bp, synthetic construct
  source 1..24: mol_type = other DNA
gacagatagtgggctaagaataac

SEQ ID NO: 27 - DNA, 18 bp, synthetic construct
  source 1..18: mol_type = other DNA
cggtacaacttagccacc

SEQ ID NO: 28 - DNA, 18 bp, synthetic construct
  source 1..18: mol_type = other DNA
cggtacaacttagccacc

SEQ ID NO: 29 - DNA, 20 bp, synthetic construct
  source 1..20: mol_type = other DNA
ccaccaactccttgagaatg

SEQ ID NO: 30 - DNA, 25 bp, synthetic construct
  source 1..25: mol_type = other DNA
gatagtgggctaagaataactttgc

SEQ ID NO: 31 - DNA, 20 bp, synthetic construct
  source 1..20: mol_type = other DNA
ccaactccttgagaatgacc

SEQ ID NO: 32 - DNA, 23 bp, synthetic construct
  source 1..23: mol_type = other DNA
ccttaatagttcattgtcgagcg

SEQ ID NO: 33 - DNA, 25 bp, synthetic construct
  source 1..25: mol_type = other DNA
gacaagcttaattacctaacagagg

SEQ ID NO: 34 - DNA, 96 bp, synthetic construct
  source 1..96: mol_type = other DNA
cctaccttagggactagtggaggcggagggggcagcggaggcggaggggggagtgggggcggcggcggtagcactagtggaattgacctggaaccc

SEQ ID NO: 35 - protein, 32 aa, synthetic construct
PTLGTSGGGGGSGGGGGSGGGGGSTSGIDLEP

SEQ ID NO: 36 - DNA, 4713 bp, synthetic construct
  source 1..4713: mol_type = other DNA
agatctgacctgggtagtgggcaatgctgtagaacaagcgtttgagctactggtggaacagttaaatcaggtaaagggtttaacagttaatttagcgcccttaaacagtgactattggggtcaggaaattacggtgacgggactattgacggggcaggatttaatcgctaaattggcaggcagagatttaggggatggtattctattgcctgctctgatgttgaaacatgatgatactcgcttcttggatgacctccgggtggccgatgtggctcaaaagttgggcacaaccatttaccctgtggctgatgtggccagtttgttggaacattgtgttcagcccatggctgtctcccgccattgctaaaaaaccccgttaatttttcttacccatggggaagtttgctggttcaataggccgagttgtagtcggcagtctatcttgagaacaattgtttttacatgacagatagtgggctaagaataactttgctcaaaccatttggaaaatcaaaggatcttcttgagatcctttttttgaattcttgacaattaatcatccggctcgtataatgtgtggaattgtgagcggataacaatttcacacaggagatataatatgagaaaccctgatactttggggctgtggacgaaaaccatggttgctctgcgccgcttcacggtgttggcgatcgccacagtcagcgtctttttgatcaccgaccttggtctgccccaggccgcctctgcctatcccttttgggcccaggaaacagcgcctttgaccccccgggaagccactggccgtattgtttgtgctaactgccacctcgcccagaaagcggcagaagtggaaattccccaggcagttttgccagacaccgtgtttgaagcagtagtcaaaattccctatgatctcgatagtcagcaggtactaggagatggttccaagggtggcttaaacgttggagccgtgttgatgttgcccgaagggttcaaaattgctccccccgatcgcctgtccgagggacttaaagaaaaagttggcggcacctacttccagccctaccgggaagacatggaaaatgtggtcattgttggccccctgcccggtgagcagtatcaggaaatcgttttccccgtgctatcccccgatcctgccaaggataaaagcattaactacggtaaattcgctgtccacctgggggccaaccggggccgtggacaaatttatcccactggtttgctgagcaataacaatgcctttaaagcccccaacgctggcaccatttcagaagttaacgcccttgaagctggtggttaccaactgatcctcaccactgccgacggtacagaaaccgtggacattcccgccggccccgaattgattgtcagtgccggtcaaactgtggaagctggggaatttttgaccaataatcccaatgtgggcggttttggccagaaagatacggaagtagtgctgcaaaatcccacccgcattaaattccttgtactcttcttggcggggattatgctctcccaaattctgttggtgttgaagaaaaaacagattgaaaaagttcaagctgccgaacttaatttctagtctagaactagtagcggccgctgcagccaggcatcaaataaaacgaaaggctcagtcgaaagactgggcctttcgttttatctgttgtttgtcggtgaacgctctctactagagtcacactggctcaccttcgggtgggcctttctgcgtttatacgagttgatcgggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagagcgttgctcaatgtacctataaccagaccgttcagctggatattacggcctttttaaagaccgtaaagaaaaataagcacaagttttatccggcctttattcacatccttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgcctctggcgattcaggttcatcatgccgtttgtgatggcttccatgtcggcagaatgcttaatgaattacaacagtactgcgatgagtggcagggcggggcgtaatttgatatcgagctcgcttggactcctgttgatagatccagtaatgacctcagaactccatctggatttgttcagaacgctcggttgccgccgggcgttttttattggtgagaatccaagcctcgagctgggatccgtggtcattctcaaggagttggtggctaagttgtaccgggaacaaaataaagtgcaggatctgttgggggccatgggctacgccctgcggagtttacataatctgaatcagtttctggaattgacccctctgatggccaccagggtcaccgatgccgatggcagtgctttagttctaatgagagagggggaaatatctatctttgaacaaatccatggccacaaaaatagtcttaagggcaccattaaaggcgctttgcagaaggcccgccaggttaacctcacattggattcttccactgtcctgagttattttgaccgtcaattgcgccaagaattaccggcgatcgcctgttataacacccccattttgagtcaccaagaggaagtgggccgcttatatatctttagtcaagatcgtaactatagctggactcccacccgtcgtaagttactacagctaatttctgatcaaaccgctgtggcgatcgccaatagtgagtcgacaaaagaagaccatatacgtctcctataggtctcggcagagcattacgctgacttgacgggacggcgcaagctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaggcgagagtagggaactgccaggcatcaaactaagcagaaggcccctgacggatggcctttttgcgtttctacaaactctttctgtgttgtaaaacgacggccagtcttaagctcgggccccctgggcggttctgataacgagtaatcgttaatccgcaaataacgtaaaaacccgcttcggcgggtttttttatggggggagtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatgcatttagaatacgtactcagcgtctgggttccccatcggtgatgtcgtataagagacgtataggagacctatagtgtcttcgggg

SEQ ID NO: 37 - DNA, 4395 bp, synthetic construct
  source 1..4395: mol_type = other DNA
agatctgacctgggtagtgggcaatgctgtagaacaagcgtttgagctactggtggaacagttaaatcaggtaaagggtttaacagttaatttagcgcccttaaacagtgactattggggtcaggaaattacggtgacgggactattgacggggcaggatttaatcgctaaattggcaggcagagatttaggggatggtattctattgcctgctctgatgttgaaacatgatgatactcgcttcttggatgacctccgggtggccgatgtggctcaaaagttgggcacaaccatttaccctgtggctgatgtggccagtttgttggaacattgtgttcagcccatggctgtctcccgccattgctaaaaaaccccgttaatttttcttacccatggggaagtttgctggttcaataggccgagttgtagtcggcagtctatcttgagaacaattgtttttacatgacagatagtgggctaagaataactttgctcaaaccatttggaaaatcaaaggatcttcttgagatcctttttttgaattcttgacaattaatcatccggctcgtataatgtgtggaattgtgagcggataacaatttcacacaggagatataatatgttttcaaaagaagtcaccgaatcaaaagtttttcaatggttcaatgatcgtctggaagtgcaagccatctccgatgacattgccagcaaatacgttcctccccacgtcaatattttttactgtctggggggcctgaccctgacctgcttcctgatccagtttgccactgggttcgccatgaccttctactacaaacccacggtcacggaagccttcgcctctgtccaatacatcatgaatgaagtcaactttggttggctgattcgctccattcaccgttggtccgccagcatgatggtgctgatgatgattctccacgtttttcgggtttacctcaccggtggtttcaagaagccccgggaattgacctgggttgtgggtgtaatgctagccgtcaccactgtcacctttggtgtaaccggttactcccttccctgggaccaggtgggttactgggcggtaaaaatcgtttccggtgtaccggccgctattcccgtggttggggatcaattggtgaccctcatgcgaggtagtgaaagcgttggtcaggctaccttgacccgcttctacagcctccacaccttcgtgcttccctgggcgatcgccgtgttgctgttgttgcacttcctgatgatccgcaaacaaggcatttccggtcctttgtaatctagaactagtagcggccgctgcagccaggcatcaaataaaacgaaaggctcagtcgaaagactgggcctttcgttttatctgttgtttgtcggtgaacgctctctactagagtcacactggctcaccttcgggtgggcctttctgcgtttatacgagttgatcgggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagagcgttgctcaatgtacctataaccagaccgttcagctggatattacggcctttttaaagaccgtaaagaaaaataagcacaagttttatccggcctttattcacatccttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgcctctggcgattcaggttcatcatgccgtttgtgatggcttccatgtcggcagaatgcttaatgaattacaacagtactgcgatgagtggcagggcggggcgtaatttgatatcgagctcgcttggactcctgttgatagatccagtaatgacctcagaactccatctggatttgttcagaacgctcggttgccgccgggcgttttttattggtgagaatccaagcctcgagctgggatccgtggtcattctcaaggagttggtggctaagttgtaccgggaacaaaataaagtgcaggatctgttgggggccatgggctacgccctgcggagtttacataatctgaatcagtttctggaattgacccctctgatggccaccagggtcaccgatgccgatggcagtgctttagttctaatgagagagggggaaatatctatctttgaacaaatccatggccacaaaaatagtcttaagggcaccattaaaggcgctttgcagaaggcccgccaggttaacctcacattggattcttccactgtcctgagttattttgaccgtcaattgcgccaagaattaccggcgatcgcctgttataacacccccattttgagtcaccaagaggaagtgggccgcttatatatctttagtcaagatcgtaactatagctggactcccacccgtcgtaagttactacagctaatttctgatcaaaccgctgtggcgatcgccaatagtgagtcgacaaaagaagaccatatacgtctcctataggtctcggcagagcattacgctgacttgacgggacggcgcaagctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaggcgagagtagggaactgccaggcatcaaactaagcagaaggcccctgacggatggcctttttgcgtttctacaaactctttctgtgttgtaaaacgacggccagtcttaagctcgggccccctgggcggttctgataacgagtaatcgttaatccgcaaataacgtaaaaacccgcttcggcgggtttttttatggggggagtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatgcatttagaatacgtactcagcgtctgggttccccatcggtgatgtcgtataagagacgtataggagacctatagtgtcttcgggg

SEQ ID NO: 38 - DNA, 4305 bp, synthetic construct
  source 1..4305: mol_type = other DNA
agatctgacctgggtagtgggcaatgctgtagaacaagcgtttgagctactggtggaacagttaaatcaggtaaagggtttaacagttaatttagcgcccttaaacagtgactattggggtcaggaaattacggtgacgggactattgacggggcaggatttaatcgctaaattggcaggcagagatttaggggatggtattctattgcctgctctgatgttgaaacatgatgatactcgcttcttggatgacctccgggtggccgatgtggctcaaaagttgggcacaaccatttaccctgtggctgatgtggccagtttgttggaacattgtgttcagcccatggctgtctcccgccattgctaaaaaaccccgttaatttttcttacccatggggaagtttgctggttcaataggccgagttgtagtcggcagtctatcttgagaacaattgtttttacatgacagatagtgggctaagaataactttgctcaaaccatttggaaaatcaaaggatcttcttgagatcctttttttgaattcttgacaattaatcatccggctcgtataatgtgtggaattgtgagcggataacaatttcacacaggagatataatatgttagtaaaaatccttaaatttagacggttcattatgacacagatttctggctcccctgatgtccccgatttgggtcggcgtcagtttatgaacttattgaccttcggtacgatcactggggtggcggccggggccctctatccagcagtaaaatatttgatccccccctccagtggtggttcgggcggtggtgtaacggccaaagatgcccttggtaatgatgttaaggtgacggaatttttggcttcccataacgctggcgatcgggtgttggcccaggggttgaagggcgacccgacctatattgtagtccagggggatgacacgatcgccaactatggtattaatgcggtgtgtacccatttgggctgtgtagtgccctggaatgccagcgaaaacaaatttatgtgcccttgtcacggctcccagtacaacgctgaaggtaaagtggtccgtggcccggctcccctctctttggctttggcccacgctaccgttaccgacgacgacaaattagtgctcagcacctggaccgaaaccgatttccgcaccgacgaagatccctggtgggcttagtctagaactagtagcggccgctgcagccaggcatcaaataaaacgaaaggctcagtcgaaagactgggcctttcgttttatctgttgtttgtcggtgaacgctctctactagagtcacactggctcaccttcgggtgggcctttctgcgtttatacgagttgatcgggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagagcgttgctcaatgtacctataaccagaccgttcagctggatattacggcctttttaaagaccgtaaagaaaaataagcacaagttttatccggcctttattcacatccttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgcctctggcgattcaggttcatcatgccgtttgtgatggcttccatgtcggcagaatgcttaatgaattacaacagtactgcgatgagtggcagggcggggcgtaatttgatatcgagctcgcttggactcctgttgatagatccagtaatgacctcagaactccatctggatttgttcagaacgctcggttgccgccgggcgttttttattggtgagaatccaagcctcgagctgggatccgtggtcattctcaaggagttggtggctaagttgtaccgggaacaaaataaagtgcaggatctgttgggggccatgggctacgccctgcggagtttacataatctgaatcagtttctggaattgacccctctgatggccaccagggtcaccgatgccgatggcagtgctttagttctaatgagagagggggaaatatctatctttgaacaaatccatggccacaaaaatagtcttaagggcaccattaaaggcgctttgcagaaggcccgccaggttaacctcacattggattcttccactgtcctgagttattttgaccgtcaattgcgccaagaattaccggcgatcgcctgttataacacccccattttgagtcaccaagaggaagtgggccgcttatatatctttagtcaagatcgtaactatagctggactcccacccgtcgtaagttactacagctaatttctgatcaaaccgctgtggcgatcgccaatagtgagtcgacaaaagaagaccatatacgtctcctataggtctcggcagagcattacgctgacttgacgggacggcgcaagctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaggcgagagtagggaactgccaggcatcaaactaagcagaaggcccctgacggatggcctttttgcgtttctacaaactctttctgtgttgtaaaacgacggccagtcttaagctcgggccccctgggcggttctgataacgagtaatcgttaatccgcaaataacgtaaaaacccgcttcggcgggtttttttatggggggagtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatgcatttagaatacgtactcagcgtctgggttccccatcggtgatgtcgtataagagacgtataggagacctatagtgtcttcgggg

SEQ ID NO: 39 - DNA, 4209 bp, synthetic construct
  source 1..4209: mol_type = other DNA
agatctgacctgggtagtgggcaatgctgtagaacaagcgtttgagctactggtggaacagttaaatcaggtaaagggtttaacagttaatttagcgcccttaaacagtgactattggggtcaggaaattacggtgacgggactattgacggggcaggatttaatcgctaaattggcaggcagagatttaggggatggtattctattgcctgctctgatgttgaaacatgatgatactcgcttcttggatgacctccgggtggccgatgtggctcaaaagttgggcacaaccatttaccctgtggctgatgtggccagtttgttggaacattgtgttcagcccatggctgtctcccgccattgctaaaaaaccccgttaatttttcttacccatggggaagtttgctggttcaataggccgagttgtagtcggcagtctatcttgagaacaattgtttttacatgacagatagtgggctaagaataactttgctcaaaccatttggaaaatcaaaggatcttcttgagatcctttttttgaattcttgacaattaatcatccggctcgtataatgtgtggaattgtgagcggataacaatttcacacaggagatataatatgagtattatcaaaaagccggatcttagcgatcccgatttacgggccaaacttgccaaaggcatgggtcacaactattatggtgagcccgcctggcccaacgacattctttacatgtttcctatctgcatccttggtgctttggggctaattgccggattggcgatcctcgaccctgccatgatcggtgaaccagcggatccctttgcgacccctctggaaattttgcctgaatggtacctgtaccccactttccaaattttgcgcattcttcccaacaaacttttgggcattgccggtatggctgccattccccttggtttgatgttggtgcctttcattgaaagtgtgaacaaattccaaaaccccttccgtcgtcccattgccatgactgtcttcttgttcggcactgcggcggctttatggttaggtgctggggcaactttccccattgataagtccctaaccttgggcttgttctaatctagaactagtagcggccgctgcagccaggcatcaaataaaacgaaaggctcagtcgaaagactgggcctttcgttttatctgttgtttgtcggtgaacgctctctactagagtcacactggctcaccttcgggtgggcctttctgcgtttatacgagttgatcgggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagagcgttgctcaatgtacctataaccagaccgttcagctggatattacggcctttttaaagaccgtaaagaaaaataagcacaagttttatccggcctttattcacatccttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgcctctggcgattcaggttcatcatgccgtttgtgatggcttccatgtcggcagaatgcttaatgaattacaacagtactgcgatgagtggcagggcggggcgtaatttgatatcgagctcgcttggactcctgttgatagatccagtaatgacctcagaactccatctggatttgttcagaacgctcggttgccgccgggcgttttttattggtgagaatccaagcctcgagctgggatccgtggtcattctcaaggagttggtggctaagttgtaccgggaacaaaataaagtgcaggatctgttgggggccatgggctacgccctgcggagtttacataatctgaatcagtttctggaattgacccctctgatggccaccagggtcaccgatgccgatggcagtgctttagttctaatgagagagggggaaatatctatctttgaacaaatccatggccacaaaaatagtcttaagggcaccattaaaggcgctttgcagaaggcccgccaggttaacctcacattggattcttccactgtcctgagttattttgaccgtcaattgcgccaagaattaccggcgatcgcctgttataacacccccattttgagtcaccaagaggaagtgggccgcttatatatctttagtcaagatcgtaactatagctggactcccacccgtcgtaagttactacagctaatttctgatcaaaccgctgtggcgatcgccaatagtgagtcgacaaaagaagaccatatacgtctcctataggtctcggcagagcattacgctgacttgacgggacggcgcaagctcatgaccaaaatcccttaacgtgagttacgcgcgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttagcccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaaggcgagagtagggaactgccaggcatcaaactaagcagaaggcccctgacggatggcctttttgcgtttctacaaactctttctgtgttgtaaaacgacggccagtcttaagctcgggccccctgggcggttctgataacgagtaatcgttaatccgcaaataacgtaaaaacccgcttcggcgggtttttttatggggggagtttagggaaagagcatttgtcagaatatttaagggcgcctgtcactttgcttgatatatgagaattatttaaccttataaatgagaaaaaagcaacgcactttaaataagatacgttgctttttcgattgatgaacacctataattaaactattcatctattatttatgattttttgtatatacaatatttctagtttgttaaagagaattaagaaaataaatctcgaaaataataaagggaaaatcagtttttgatatcaaaattatacatgtcaacgataatacaaaatataatacaaactataagatgttatcagtatttattatgcatttagaatacgtactcagcgtctgggttccccatcggtgatgtcgtataagagacgtataggagacctatagtgtcttcgggg

SEQ ID NO: 40 - DNA, 12468 bp, synthetic construct
  source 1..12468: mol_type = other DNA
gagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccccgggtaccgagctcgaattacattgcaaacagtccaaaattctcagcaaatctaccagttgtgagggaacttggaactcggccccgatcgccttggggctgagccgaaaatctccatcgatcctggcccaatagccatatagcttttggttggaaaaagagctattgacggcccaacccccgtcattgaggggagttatttcggcaatggcatggggaggcacttccagttcccgatgaaggatttcagccaaagttagctccaccttagctagatctgttagccgccctccagggaaatcccaagttgccctacctatacccggccggaaaatgggatggggtaaaagcaggcgatcgccttgttggggcaacactattaaagagtcagccttttccacccgccaataatccaatttcctttcccgatcatcccgccaccgttcccccctgagggtcaaccaagggtttttaacctccgcaaaacactccagtaaagtccaagccggagcatcgggctgatttccccttgcggactctgagccaatttgtaaaacttgcccctcgttccaagggcgatcataacttttatctatggagatcattgtcaaaatccgcttcttaacatttcttaaaattaaagctgttatagcaacaacattgattaatttctatctaatttttgacggtgcccattgctatcagttgtaagttgatgaaaatgctgtaaatttttgtaacaaagttcaactttgtcttgacttttgtaagtctttgcaaaatctaggagctagaactggtcagggctggggcaatttttaattattgttacgcaggtcttgcctagggggggggaggccgtattatcttctagtgatgtttgctgaaaacgcctatctgtgcaaggtttaacatcgttattatgaagcgaaaactaattcccttttttacgcttcctctattacactattctgcataggaaacccttaatagttcattgtcgagcgaggagaaccctgcatggggggttgttgacatattcctgacccaatttaatcccaacttaattttggttaactttctttagatcccgccccgccgggatttttttgtcttatggagcatagggggccacaaactgtcattggctagtcagggaatatcgcccaaagccttgtctagatgggctggcgcgttttctgtcataatcgaacaggacttgttacattttttattgcaaatccccggggtagcggtaacttagccggcctgcctctctgtaacgatgttgccggggtttctccggtgaatgttagtaaaaatccttaaatttagacggttcattatgacacagatttctggctcccctgatgtccccgatttgggtcggcgtcagtttatgaacttattgaccttcggtacgatcactggggtggcggccggggccctctatccagcagtaaaatatttgatccccccctccagtggtggttcgggcggtggtgtaacggccaaagatgcccttggtaatgatgttaaggtgacggaatttttggcttcccataacgctggcgatcgggtgttggcccaggggttgaagggcgacccgacctatattgtagtccagggggatgacacgatcgccaactatggtattaatgcggtgtgtacccatttgggctgtgtagtgccctggaatgccagcgaaaacaaatttatgtgcccttgtcacggctcccagtacaacgctgaaggtaaagtggtccgtggcccggctcccctctctttggctttggcccacgctaccgttaccgacgacgacaaattagtgctcagcacctggaccgaaaccgatttccgcaccgacgaagatccctggtgggcttaggtttagctttgctatccgttgagtaagcaacactgttgtaattttgtgtaattgtctgagactggtctttttcccctactgttcacctaaacctaatccgttcaacttaacgatgagaaaccctgatactttggggctgtggacgaaaaccatggttgctctgcgccgcttcacggtgttggcgatcgccacagtcagcgtctttttgatcaccgaccttggtctgccccaggccgcctctgcctatcccttttgggcccaggaaacagcgcctttgaccccccgggaagccactggccgtattgtttgtgctaactgccacctcgcccagaaagcggcagaagtggaaattccccaggcagttttgccagacaccgtgtttgaagcagtagtcaaaattccctatgatctcgatagtcagcaggtactaggagatggttccaagggtggcttaaacgttggagccgtgttgatgttgcccgaagggttcaaaattgctccccccgatcgcctgtccgagggacttaaagaaaaagttggcggcacctacttccagccctaccgggaagacatggaaaatgtggtcattgttggccccctgcccggtgagcagtatcaggaaatcgttttccccgtgctatcccccgatcctgccaaggataaaagcattaactacggtaaattcgctgtccacctgggggccaaccggggccgtggacaaatttatcccactggtttgctgagcaataacaatgcctttaaagcccccaacgctggcaccatttcagaagttaacgcccttgaagctggtggttaccaactgatcctcaccactgccgacggtacagaaaccgtggacattcccgccggccccgaattgattgtcagtgccggtcaaactgtggaagctggggaatttttgaccaataatcccaatgtgggcggttttggccagaaagatacggaagtagtgctgcaaaatcccacccgcattaaattccttgtactcttcttggcggggattatgctctcccaaattctgttggtgttgaagaaaaaacagattgaaaaagttcaagctgccgaacttaatttccctaccttagggactagtggaggcggagggggcagcggaggcggaggggggagtgggggcggcggcggtagcactagtggaattgacctggaacccatgacaattagtccacccgaaagagaggctaaggccaaagtctcggttgataacaacccggtaccaacttccttcgagaagtggggcaagccgggtcacttcgaccggactttagctagaggtcccaaaaccaccacttggatttggaatctccatgccaatgcccatgattttgatagtcagaccagcgatttagaagatgtttcgcgcaaaatcttcagtgctcactttgggcacctcgctgtggtctttgtttggttaagtggaatgtacttccacggcgcgaaattttccaattacgaaggttggttagccgaccctacccacattaaacccagtgcccaagtggtttggcccattgtcggtcaaggcatcttgaacggggatgtgggcggtggcttccacggtattcagattacgtctggcctgttttatctctggcgggcctccggtttcaccgacagctatcagctctactgcaccgccattggcggtttggttatggctgccctgatgctgttcgctggctggttccactaccacgtcaaagctcccaaattggaatggttccaaaatgtggagtcgatgatgaaccatcatttggctggtttgttgggcttaggctccttgggatgggcgggtcaccagatccacgtctccatgcccattaataaacttttggatgctggggttgctcctaaggacattccccttccccacgagtttattttggaaccgagcaagatggcggaactctaccccagctttgcccaaggtttgacaccgttctttaccctcaactggggagtctactcagacttcctcacctttaaagggggattgaatccagtcaccggcggcctctggttgtctgataccgctcaccaccatttggcgatcgccgtcctgttcatcattgccggtcatatgtaccgcacgaactggggcatcggccatagcatgaaagagatcctcgaagcccacaagggccccttcactggggaaggtcacaaaggactttatgaaatcctgactacttcttggcacgctcaattagccattaacctcgccctgttaggttctttgacgatcatcgttgcacaacacatgtatgcgatgccgccctatccctaccaggcgatcgattacgctacgcagttatccctgttcacccaccacatgtggattggcggcttcctcatcgtaggggccggtgcccatggcgccatcttcatggtgcgtgattatgatcccgccaagaacgttaataacttgctggatcggatgctgcgccaccgtgacgccatcatttcccatctcaactgggtatgtattttcctcggcttccatagcttcggtctctacatccacaacgacaccatgcgggctttaggccgtccccaggacatgttctccgacacggccatccaactgcaaccgatctttgcccaatgggtacagcacctgcacaccttggcccccggagccaccgctcccaatgccttggccactgccagctatgcctttggcggagaaaccattgccgttgccggtaaagtcgccatgatgcccatcaccctgggtactgccgacttcatggttcaccacatccacgcttttaccatccacgtaacggccctgatcctcctcaaaggggttctatatgcccgtagctctcgccttgtccctgataaagcgaatctcggtttccgtttcccctgtgatggccccggccgcggcggcacctgccaagtatctggttgggaccacgttttcctcggtctgttctggatgtacaactccctttccgtcgtcatcttccacttcagttggaaaatgcaatccgatgtttggggtaccgtttctcccgatggcagtgtcacccatgtgaccctaggtaactttgcccagagtgccatcaccatcaatggctggttgcgggacttcctctgggcccaagccgccaacgtcatcaactcctacggttccgccctgtcggcctatggcatcatgttcctcgccggacactttgtctttgccttcagcctcatgttcctgttcagtggacggggatattggcaagagttgatcgagtccattgtctgggctcacaacaaactgaatgtggctccggccattcaaccccgcgctttgagcatcattcaaggtcgtgcggtcggtgtagcgcactatctcctcggaggtattgttaccacctgggcgttcttcctcgcccgcagtctttccattggctagactttgagctgaagttgggttttccgggataaaccagggggactggctgaggtttttactatccttaatcctccccctgccctccaccctccccagcatttaatagaagcggttttaatttcgcttttaccctatattcacccctataaccccttgttatagtggctttaacaatccggaaatgagcagcgcttctgcattagcctcattttgcttgcttaacgaggagaatttccgaaaaagctatggcaactaaatttcctaaatttagccaggatctcgcccaagacccgactacacggcgtatttggtacgggattgctacggcccacgactttgaaacccacgatgggatgactgaggagaatctttaccaaaagatttttgcctcccactttggacacatcgccatcattttcctgtggacgtctggcacccttttccacgttgcgtggcaaggtaattttgaacaatggattaaagatcctttaaacattcgcccgatcgcccatgcgatttgggacccccactttggcgagggagctgtcaatgctttcacccaagctggggcttctaacccggttaacattgcttattccggggtttaccactggttctacaccattggtatgaccaccaaccaagagctctattctggtgcggtcttcctattagtgttagcttccctgtttttatttgcaggctggttacacctccaaccgaagttccgtcctagcttagcctggttcaaaaatgccgaatcccgcttaaatcaccacctggctggtttgttcggggttagctccttggcttgggctggtcacttggtccacgttgcgattcccgaagcccggggtcaacacgttggttgggataacttcctgtctactcctccccaccccgctggtttgatgcctttcttcaccggcaactggggcgtgtatgcggcggatcccgacactgctggccacatttttggtacttctgaaggtgctggtaccgcaatcctgaccttcctgggtggtttccatccccaaacggaatctctctggttgacggacattgcccaccaccatttggcgatcgccgtgatcttcatcattgctggtcacatgtatcgcaccaactggggcattggccacagcatcaaagaaattcttaatgcccataaaggtcccctaaccggcgcaggccataccaacctgtacgacaccatcaataactccctccacttccaactcggcttagccttggcaagcttaggggttattacttccctggtggcgcagcacatgtactccctgccctcctacgcctttattgcccaggaccacaccacccaggcagccctctacacccatcaccagtacattgctggattcttgatggttggtgcttttgcccacggtgccatcttctttgtccgggattacgatcccgtggccaataaagataacgtgctggcccgcatgcttgaacacaaagaggctctgatttcccacttaagctgggtgtccctcttcttgggcttccacacccttggtctttatgtccataacgatgtggtggtggccttcggtactcccgaaaaacaaattctgatcgagcccgtttttgcccaatggattcaagcaacttccggtaaagctctctatggctttgatgttttgctctccaatcctgacagcattgcttccaccactggagccgcttggttacccggctggttggatgctatcaacagcggcaccaactctctgttcttgaccattggccctggcgacttcttggttcaccacgcgatcgccctagggttgcacaccactgccctgattctaatcaaaggtgctttggatgcccgtggttctaagttgatgccggacaaaaaggacttcggttactccttcccctgtgatggccccggccgtggcggtacctgcgacatctctgcttgggatgccttctacctagccatgttctggatgctgaacaccttgggttggttgaccttctactggcactggaaacacctcggtgtttggagcggtaacgttgctcagttcaacgaaaactccacctacctgatgggttggttccgggattacctctgggcgaactctgctcagttaatcaatggttacaacccctacggtgtcaacaatctgtcagtttgggcttggatgttccttttcggacacctggtctgggctactggcttcatgttcttgatctcttggcggggttactggcaagagttgattgaaaccatcgtttgggcccacgagcgcactcctttggcgaacttggttcgttggaaagataagcccgttgcgttgtccattgttcaagcccgtttggttggtttagcccacttcaccgttggttatgtgctcacctatgcggcattcctaattgcttccacagccggtaagttcggttaagtttatactcgagctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctccaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgcacgaattcctctgttaggtaattaagcttgtcccctgccctcgttggtgggggatttgctttaattggctgatcgccacaaaaaatggattggaatcggcctcatttaatcttaagttttttgttatttttaccccattgctaatttgcttccatcccaagtaggcggtcgccacagagttcgattagggcagggatatgatgatggcggttggggatgcgccaactaatggcaatatgccgcaatagagtaagaatgatgtaaatatcaatggcttggctaagatggggattatttggggattgtttagctagaaaagcagtttttaatatttgttctttgcgaactagggcatcgggattcccccaggtgcggaggctatattcggtcatgtgggcgataaagtttcctagatcaacggcgggatctcctttgcagtagaggtcgaggtccaccaaccaaaggtgattgccatctaccaaaatttgatcggggtaaaaatcccggtgaatagtggtagtaggacggggatatttgtttaaaacctctcccagctgttcactctgggcgatgaaatgttgaatttgtgtttgccactgaggttgctggtggctaaattcccggaggcgatcgccaaggatttgtaattcagtggcaatggtatgggttttggttgtggggacagaatggctgtggattttgtgggctagggcggcaattttttccggtaagtcgggacgggtaatcaataattctgtcgcaggttgaccgggaacccatcgttggagccacatctgccagggttcaacaatgcccaaaggttttggcaccgataatccatcttcactttgcccatggaacccgttttcccataacgcccgttgagagccatagcttttgtaatctgtgcccttggcgcgaattttgccaagaataattccttccaccccatcggtttccagatgataggcaatcaatgcccgtcgcccaggtttatggcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaact

SEQ ID NO: 41 - protein, 15 aa, synthetic construct
PTLGMGGGGGSDLEP

SEQ ID NO: 42 - protein, 21 aa, synthetic construct
PTLGMGGGGGSGGGGGSDLEP

SEQ ID NO: 43 - protein, 27 aa, synthetic construct
PTLGMGGGGGSGGGGGSGGGGGSDLEP

(19) United States
(12) Patent Application Publication
(10) Pub. No.: US 2025/0340829 A1
(43) Pub. Date: Nov. 6, 2025

(54) ENGINEERING PHOTOSYNTHETIC ELECTRON TRANSPORT CHAIN FOR IMPROVED PHOTOSYNTHETIC EFFICIENCY

(71) Applicants: Arul Mozhy Varman, Chandler, AZ (US); Abhishek Singharoy, Chandler, AZ (US); Willem Vermaas, Tempe, AZ (US); Aditya Sarnaik, Tempe, AZ (US); Nima Hajinajaf, Tempe, AZ (US)

(72) Inventors: Arul Mozhy Varman, Chandler, AZ (US); Abhishek Singharoy, Chandler, AZ (US); Willem Vermaas, Tempe, AZ (US); Aditya Sarnaik, Tempe, AZ (US); Nima Hajinajaf, Tempe, AZ (US)

(73) Assignee: Arizona Board of Regents on Behalf of Arizona State University, (omitted)

(21) Appl. No.: 19/196,259

(22) Filed: May 1, 2025

Related U.S. Application Data
(60) Provisional application No. 63/641,381, filed on May 1, 2024.

(51) Int. Cl. C12N 1/20; C12N 15/74; C12R 1/01

(52) U.S. Cl. CPC C12N 1/20; C12N 15/74; C12N 2800/101; C12N 2830/008; C12N 2830/20; C12R 2001/01

(57) ABSTRACT

Methods of engineering a photosynthetic bacteria or a plant to improve their photosynthetic efficacy by modifying a photosynthetic electron transport chain are disclosed herein. The method may comprise engineering the photosynthetic bacteria or the plant to overexpress a cyt b₆f major protein or to express a synthetic construct of cyt b₆f major protein linked to a photosystem protein. The engineered organisms exhibit increased growth as a result of increased photosynthetic efficiency. The disclosure also relates to methods of increasing biomanufacturing by the photosynthetic bacteria or plant, for example increased production of a biofuel, a biofertilizer, a nutraceutical, or a pharmaceutical.

### RELATED APPLICATIONS

This application claims priority to and the benefit of U.S. provisional patent application 63/641,381, filed May 1, 2024 to Varman et al., titled “ENGINEERING PHOTOSYNTHETIC ELECTRON TRANSPORT CHAIN FOR IMPROVED PHOTOSYNTHETIC EFFICIENCY,” the entirety of the disclosure of which is hereby incorporated by reference.

### STATEMENT REGARDING FEDERALLY SPONSORED RESEARCH OR DEVELOPMENT

This invention was made with government support under 2146114 awarded by the National Science Foundation. The government has certain rights in the invention.

### SEQUENCE LISTING

In accordance with 37 C.F.R. § 1.831, the present specification makes reference to a Sequence Listing submitted electronically in the form of an XML file (entitled “11157-177SeqList.xml”, created on Apr. 29, 2025, 68,437 bytes in size). The entire contents of the Sequence Listing are herein incorporated by reference in their entirety, with the intention that, upon publication (including issuance), this incorporated Sequence Listing will be inserted in the published document immediately before the claims.

### TECHNICAL FIELD

The present disclosure relates to engineering the photosynthetic electron transport chain to improve photosynthetic efficiency.

### BACKGROUND

Much of the energy used in fundamental cellular processes for the majority of life on Earth is provided through photosynthesis, followed by respiration via the food web. Broadly, there are two types of phototrophs, oxygenic and anoxygenic. Increasing the productivity of oxygenic photosynthesis is one approach that could be effective to improve the production efficiencies of the photoautotrophs.

Generally, photosynthetic reactions called light dependent reactions taking place within the thylakoid membranes (a cellular compartment) are comprised of a cascade of reactions viz. the light-driven splitting of water, the electron transport across the photosystems and cytochrome complexes, generation of NADPH (the cell's redox currency), and the synthesis of ATP (the energy currency of life). During the process of photosynthesis, oxygen can be produced by oxygenic phototrophs such as cyanobacteria, green algae, diatoms and plants, by utilizing water as the electron donor. The visible range of the sun's light spectrum (400-700 nm) is the primary area of solar energy absorption for photosynthesis, by the oxygenic phototrophs. Photosystem I (PSI), Photosystem II (PSII), Adenosine triphosphate (ATP) synthase, and the Cytochrome b₆f (Cyt b₆f) complex are the four fundamental components of oxygenic photosynthesis. Both the photosystems (PSI and PSII) are required for photosynthesis and include an electron transport mechanism in the thylakoid membranes. The primary differentiating factor between prokaryotic (cyanobacterial) and eukaryotic (microalgae, plants) is their light harvesting accessory protein complexes. Eukaryotes possess light harvesting complexes (LHCs), whereas cyanobacteria have phycobilisome complexes. The phycobilisome complexes work as antenna pigments for capturing the photons and exciting the reaction center chlorophylls in the PSI and PSII, resulting in an electron flow between the photosystems across cyt b₆f. Hence, despite the differences in light harvesting, cyanobacterial photosynthetic electron transport chain is equivalent to those of the phototrophic eukaryotes. These electron transport activities produce NADPH, a powerful reducing equivalent that may be utilized to assimilate inorganic minerals or for biosynthetic purposes.

Light photons excite the reaction centers in PSII (Chl-a P₆₈₀), to drive water splitting into protons, electrons, and molecular oxygen. The electrons are channeled through PSII→Cyt b₆f→PSI for the generation of NADPH. Reduced NADP (NADPH) is used to fix atmospheric CO₂ to organic carbon through light independent reaction pathways. Additionally, a proton gradient created across the thylakoid membrane, during the electron transport, and by water splitting at PSII drives the ATP synthase complex, thereby generating ATP for the light-independent reactions. Although this process is extremely robust, only 3-6% of the total solar energy harvested is conserved in biomass. However, efficiency of a photosynthetic system is predominantly affected by electron leakage during the electron transport chain. The present disclosure provides compositions, methods, and systems for a photosynthetic system that is more modular than the natural one and less amenable for electron leakage. Additionally, the present disclosure demonstrates the effects of an engineered photosynthetic system on cell growth and photosynthesis.

Solar energy harvested by cyanobacteria is a primary source for energy. Cyanobacteria has been used in diverse industries including biofuel, biofertilizer, food, nutraceuticals, and pharmaceuticals manufacturing. Therefore, it is imperative to increase its photosynthetic efficiency to secure our food supply. In exemplary embodiments of the present disclosure, the cyanobacterium Synechocystis sp. PCC 6803 (hereafter, Synechocystis) is used as a model organism as it is easy to engineer and shares all energy bottlenecks ubiquitous to photosynthesis and respiration in plant cells.

### SUMMARY OF THE DISCLOSURE

Disclosed herein are two distinct strategies to engineer the photosynthetic electron transport chain in Synechocystis to increase the photosynthetic efficiency. In some aspects, the engineered photosynthetic bacterium or plant exhibits increased photosynthetic efficiency compared to its native counterpart. As a result, the engineered photosynthetic bacterium or plant has increased cell growth and/or biomass production compared to its native counterpart.

In some embodiments, the method comprises engineering the photosynthetic bacterium or plant to overexpress a cyt b₆f major protein. In yet other implementations, the engineering step comprises transforming the photosynthetic bacterium or a cell of the plant to express the cyt b₆f major protein using an overexpression promoter, for example, a trc promoter.

In other embodiments, the method comprises engineering the photosynthetic bacterium or plant to express a synthetic construct of a cyt b₆f major protein linked to a photosystem protein. In some implementations, the engineering step comprises transforming the photosynthetic bacterium or the cell of the plant to express a plasmid expressing a cyt b₆f major protein linked to a photosystem protein. In some aspects, the operon expressing the photosystem protein in the genome of the transformed photosynthetic bacterium is replaced with a nucleotide sequence expressing the cyt b₆f major protein linked to the photosystem protein, which in some embodiments is a protein from Photosystem I or a protein from Photosystem II.

In certain implementations of the step of transforming the photosynthetic bacterium or the cell of the plant to express a plasmid expressing a cyt b₆f major protein linked to a photosystem protein, the plasmid comprises a first nucleotide sequence encoding petC:petA operon; a second nucleotide sequence encoding a linker sequence; and a third nucleotide sequence encoding a psaA-psaB operon. In particular embodiments, the second nucleotide sequence comprises the sequence set forth in SEQ ID NO. 34 or the linker sequence comprises an amino acid sequence set forth in SEQ ID NO. 35. In some aspects, the plasmid encodes a contiguous translation of any one of PetC, PetA, PsaA, PsaB, and/or a combination thereof. In certain implementations, the petC:petA operon lacks the sequence encoding a stop codon of petA, wherein the stop codon is replaced by the second nucleotide sequence thus resulting in the expression of petA linked to psaA. In some aspects, the plasmid further comprises a fourth sequence encoding a petC promoter, wherein the petC promoter controls transcription of petC, petA, psaA, and psaB. In some implementations, the plasmid comprises a fifth nucleotide sequence and a sixth nucleotide sequence that are complementary to sequences flanking either and/or both of the 5′ and 3′ termini of the psaA-psaB operon of a cyanobacteria genome. In particular implementations, the fifth nucleotide sequence and the sixth nucleotide sequences are each about 1 kb and are complementary to about a 1 kb sequence flanking either and/or both of the 5′ and 3′ termini of the psaA-psaB operon of the cyanobacteria genome.

In particular implementations of the step of engineering the photosynthetic bacterium or plant to express a synthetic construct of a cyt b₆f major protein linked to a photosystem protein, the synthetic construct comprises a linker sequence having an amino acid sequence set forth in SEQ ID NO. 35, wherein the linker sequence links the cyt b₆f major protein to the photosystem protein.

In another aspect, an engineered cyanobacterium is disclosed, for example a Synechocystis species. In some implementations, the engineered cyanobacterium overexpresses a cyt b₆f major protein, for example, PetD. In some embodiments, gene expression of the cyt b₆f major protein in the engineered cyanobacterium is at least under the regulation of a trc promoter (P_{trc}). In other implementations, the engineered cyanobacterium expresses a synthetic construct of cyt b₆f major protein linked to a photosystem protein. In some aspects, the synthetic construct comprises the cyt b₆f major protein linked to the protein from Photosystem I. In such embodiments, the synthetic construct is expressed via a modified psaA-psaB operon region comprising a first nucleotide sequence encoding petC:petA operon; a second nucleotide sequence encoding a linker sequence; and a third nucleotide sequence encoding a psaA-psaB operon. In some implementations, the modified psaA-psaB operon region further comprises a fourth sequence encoding a petC promoter that controls transcription of petC, petA, psaA, and psaB.

Accordingly, engineering photosynthetic bacteria or plant through the strategies described above to increase photosynthetic efficiency increases biomanufacturing by the photosynthetic bacteria or plant. In some aspects, the engineered photosynthetic bacterium or plant has increased production of a biofuel, a biofertilizer, a nutraceutical, or a pharmaceutical.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Implementations will hereinafter be described in conjunction with the appended and/or included DRAWINGS, where like designations denote like elements.

The features and advantages of the present disclosure, and the manner of attaining them, will become more apparent and the present disclosure will be better understood by reference to the description of the present disclosure taken in conjunction with the accompanying drawings, wherein:

FIGS. 1A and 1B depict, in accordance with certain embodiments, the design for two distinct strategies to engineer the photosynthetic electron transport chain in Synechocystis 6803. FIG. 1A depicts the strategy of overexpressing Pet subunits that are under the control of P_{trc} promoter. FIG. 1B depicts the strategy of linking PetA and PsaA.

FIG. 2 depicts, in accordance with certain embodiments, genetic constructs for integrating at genomic neutral sites.

FIG. 3 depicts, in accordance with certain embodiments, arrangements for producing Linker strains.

FIG. 4 depicts, in accordance with certain embodiments, a protein linking strategy.

FIG. 5 depicts, in accordance with certain embodiments, an in silico simulation showing the arrangement of PSI and cyt b6f complxes in the thylakoid membranes, highlighting their PsaA (Orange) and PetA (Cyan) proteins, respectively. The blue spheres indicate N terminui of the peptides and red spheres indicate C termini. Overall, the computational simulation showed that the N terminus of PsaA is in natural physical proximity with C terminus of PetA. Therefore, linking these proteins as PetA followed by PsaA can bring both the proteins and their respective photosynthetic complexes closer than natural.

FIGS. 6A and 6B depict, in accordance with certain embodiments, computational modeling of cyanobacterial super-complex. Fusion candidates were determined from a modeled cyanobacterial supercomplex from PSI (PsaA subunit colored in orange) and cytochrome b₆f (blue), with this model being based off microalgae Chlamydomonas reinhardtii supercomplex (FIG. 6A). Distance from N (blue) and C (red) termini between PsaA in PSI (orange) and PetA in cytochrome b₆f (cyan) is shown in FIG. 6B. The distance is ˜47 Å and is indicated by the dashed white line.

FIG. 7 depicts, in accordance with certain embodiments, a schematic representation of homologous recombination strategy for replacing the natural psaA:psaB locus by the engineered cargo possessing petC:petA-linker-psaA:psaB, regulated by upstream sequence of petC gene. The upstream and downstream sequences of natural psaA:psaB were used as the homologous arms. The polynucleotide and polypeptide segments of the linker peptide are in yellow and blue colors and are sequences are set forth as SEQ ID NO. 34 and SEQ ID NO. 35, respectively.

FIG. 8 depicts the map of the pFGN2 plasmid.

FIGS. 9A and 9B depict, in accordance with certain embodiments, a schematic representation of the genetic engineering strategy for overproduction of cyt b₆f major proteins. FIG. 9A depicts the pEERM4 plasmid containing the neutral site regions for homologous recombination at NS-1 in Synechocystis genome, which was used in the functional studies. Its P_{nrsb} promoter was replaced with Pre through restriction ligation, for facilitating constitutive expression.

FIG. 9B shows the genes amplified from Synechocystis genome were cloned under Pure promoter between XbaI and SpeI restriction sites, and the plamids were individually transformed constructing four different engineered strains of Synechocystis.

FIGS. 10-13 depict the maps of the pBN05, pBN06, pBN07, and pBN08 plasmids.

FIG. 14 depicts, in accordance with certain embodiments, the growth of PetD, Linker and wild-type PCC 6803 (control) strains under 250 rpm, atmospheric CO₂, and 20 μmol of photons m⁻²s⁻¹. The strain expressing PetD and Linker respectively exhibited 52% and 39% increase in cell density compared to WT at day 12.

FIG. 15 depicts, in accordance with certain embodiments, the growth of PetD, Linker and wild-type PCC 6803 (control) strains under 250 rpm, atmospheric CO₂, and moderate light (80 μmol of photons m⁻²s⁻¹). The strain expressing PetD and Linker respectively exhibited 34% and 15% increase in cell density compared to WT at day 11.

FIG. 16 depicts, in accordance with certain embodiments, the growth of PetD, Linker and wild-type PCC 6803 (control) strains under 1% CO₂ and moderate light (80 μmol of photons m⁻²s⁻¹). WT strain and the strain expressing Linker should similar growth, while the straing expressing PetD exhibited 24% higher cell density compared to WT at day 9.

FIGS. 17A-17D compare, in accordance with certain embodiments, PetD, Linker and wild-type PCC 6803 (control) strains grown large cultures (300 mL) under dark conditions (20 μmol of photons m⁻²s⁻¹), which favor PetD engineering. FIG. 17A compares the growth of the strains. FIG. 17B compares O₂ evolution of the strains. FIG. 17C compares chlorophyll a content of the strains. FIG. 17D compares carotenoids content of the strains.

FIGS. 18A-18D compare, in accordance with certain embodiments, PetD, Linker and wild-type PCC 6803 (control) strains grown under atmospheric CO₂ and high light (150 μmol of photons m⁻²s⁻¹). FIG. 18A compares the growth of the strains. The result shows longer exponential phase for the engineered strains that is mainly due to higher electron transport in those cells. FIG. 18B shows that cells turn yellow under high light exposure due to the increased production of carotenoids to confront the availability of reactive oxygen species in the cell. FIG. 18C compares chlorophyll a content of the strains. As condition was favored more for the engineered strains, the chlorophyll content was higher as opposed to control (wild-type). FIG. 18D compares carotenoids content of the strains.

FIG. 19 depicts, in accordance with certain embodiments, the rereduction rate of PetD, Linker and wild-type 6803 strains. Joliot-type spectroscopy was used to study the rereduction rate for comparing wild-type, PetD and Linker cells, absorbance at 680 nm gauged PSI's rereduction. Results showed consistent PSII and b6f inhibition patterns across all samples. Genetically engineered strains, especially PetD, exhibited significantly faster rereduction than the WT at various light intensities. PetD cells maintained accelerated rereduction even at higher light intensities, indicating enhanced electron flux through the b6f complex. Linker cells also demonstrated faster rereduction at lower intensities but were surpassed by WT at light intensities above 150 μE. Notably, at 20 μE, PetD cells rereduced 67 times faster than WT, and at 45 HE, both PetD and Linker strains rereduced at double the rate of WT. At 80 and 150 μE, Linker cells continued to rereduce faster than WT, at 35% and 15% respectively.

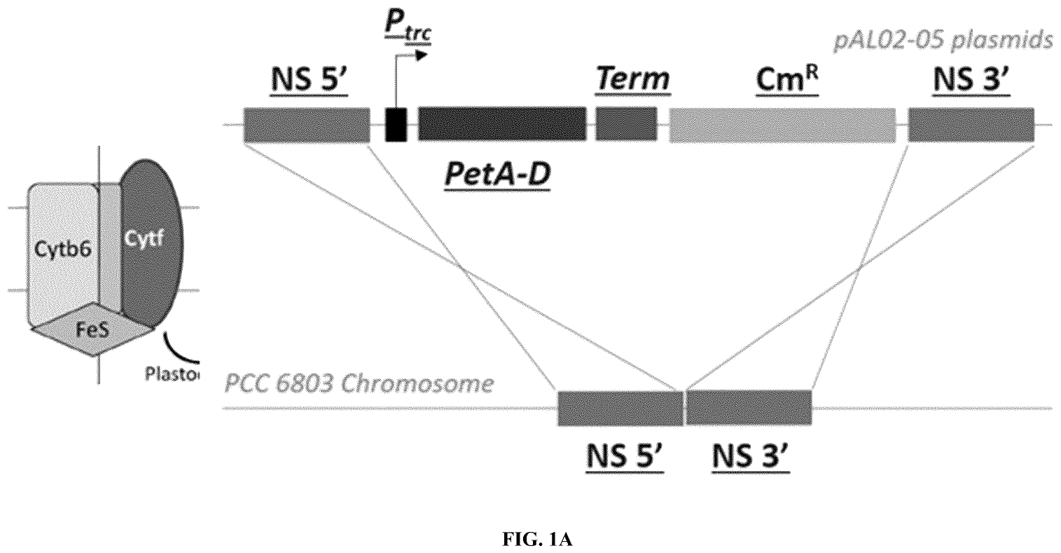

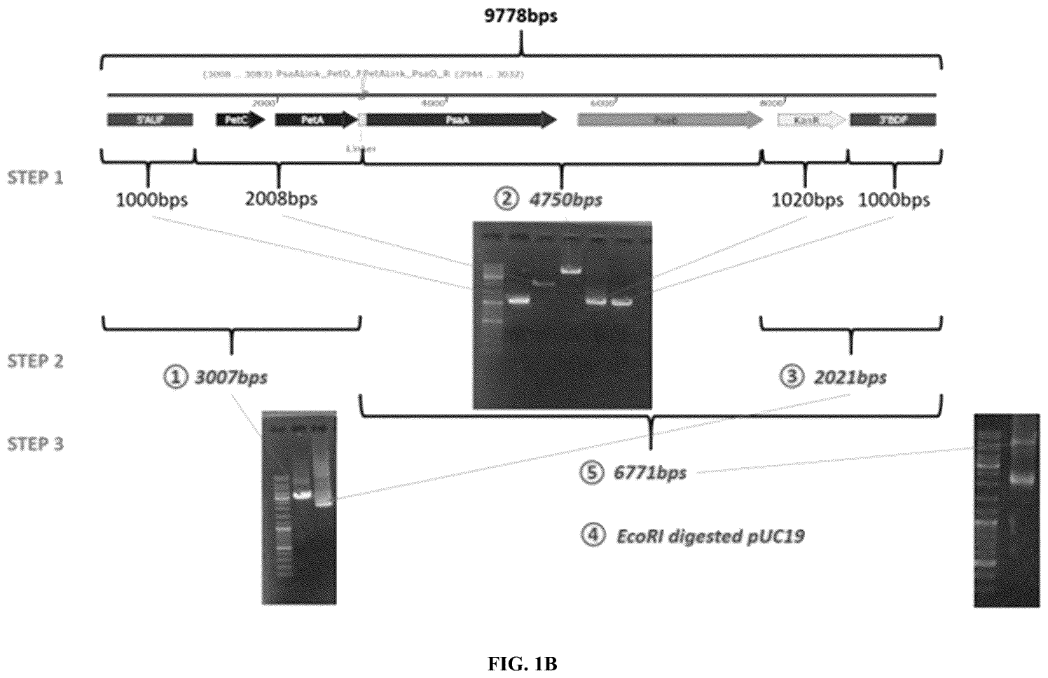

Those of ordinary skill in the art will understand that the compositions, methods, and systems specifically described herein and illustrated in the accompanying drawings are non-limiting exemplary embodiments and that the scope of the various embodiments of the present disclosure is defined solely by the claims. The features illustrated or described in connection with one exemplary embodiment may be combined with the features of other embodiments. Such modifications and variations are intended to be included within the scope of the present disclosure.

### DETAILED DESCRIPTION

Detailed aspects and applications of the disclosure are described below in the drawings and detailed description of the disclosure. Unless specifically noted, it is intended that the words and phrases in the specification and the claims be given their plain, ordinary, and accustomed meaning to those of ordinary skill in the applicable arts.

In the following description, and for the purposes of explanation, numerous specific details are set forth to provide a thorough understanding of the various aspects of the disclosure. It will be understood, however, by those skilled in the relevant arts, that the present disclosure may be practiced without these specific details. It should be noted that there are many different and alternative configurations, devices, and technologies to which the disclosed embodiments may be applied. The full scope of the disclosures is not limited to the examples that are described below.

The singular forms “a,” “an,” and “the” include plural referents unless the context clearly dictates otherwise. Thus, for example, reference to “a step” includes reference to one or more of such steps. The term “about” when used in the context of a given numerate value or range refers to a value or range that is within 10%, within 5%, within 4%, within 3%, within 2%, or within 1% of the given value or range.

There have been many different components of oxygenic photosynthesis that have been identified as targets for increasing the efficiency of photosynthesis, such as, reducing light-harvesting antenna size, introducing components of algal CO₂-concentrating mechanisms, engineering of photo-respiratory bypasses, and accelerating recovery from photo-oxidation. However, susceptibility to leakage in electron transport within the photosynthetic apparatus (PSII+b₆f+PSI+ATP synthase complex) is one of the major factors for reduced photosynthetic efficiency in phototrophs.

This disclosure provides methods, compositions, and systems facilitating effective electron channeling through the photosynthetic electron transport chain to reduce leakage and improve photosynthetic efficiency, which ultimately improves biomass product, especially for scaled-up algae cultivation systems like flat panel photobioreactors and raceway ponds. In some embodiments, effective electron channeling may be induced by clustering all or the adjoining protein complexes into a super-complex in the thylakoid membrane. In some embodiments, smoother channeling of the electrons may be induced by individually overproducing cyt b₆f major proteins thereby increasing their inter-complex density. In some such embodiments, individually overproducing cyt b₆f major proteins links the photosynthetic complexes photosystem I (PSI) and cytochrome bf (cyt b₆f), bringing them in a closer proximity and thereby, reducing electron loss. In some embodiments, smoother channeling of the electrons may be induced by physically linking cyt b₆f to the adjoining photosystems (PSI and/or PSII) thereby pulling them in physical proximity. In some such embodiments, physically linking cyt b₆f to the adjoining photosystems increases the membrane density of cyt b₆f complex and thereby reduces the proximity between cyt b₆f with PSI and PSII. In some embodiments, methods of increasing biomanufacturing by photosynthetic bacteria or a plant are described.

In some aspects, the methods increase photosynthetic efficiency of the engineered photosynthetic bacteria or plant compared to the native counterpart. In exemplary embodiments, comparative growth studies of the engineered strains with wild type under atmospheric CO₂ showed that at low light (20 μE m⁻² s⁻¹), there was a 52% and 39% increase in cell density of the strain expressing PetD and Linker compared to the WT at day 12, respectively. Interestingly, this difference in growth at moderate light (80 μE m⁻² s⁻¹) was 34% and 15% for strain expressing PetD and Linker compared to WT at day 11. Significant growth was observed in strains cultivated under high light conditions (150 μE m⁻² s⁻¹) with both PetD and Linker strains displaying an extended growth period. By day 16, this resulted in a 61% increase in growth for the PetD strain and a 74% increase for the Linker strain. Further analysis under higher CO₂ also showed % 24.7 increase in PetD growth compared to wild-type. The Juliot type spectroscopy performed to test the rereduction rate of engineered strains. The test results showed that PetD cells rereduced 67 times faster than WT at 20 μE m⁻² s⁻¹. Besides, The PetD and linker rereduced at double the rate of wildtype at 45 μE m 2 s 1 and as the light intensity increased, the PetD cells rereduction maintained faster than that of WT, approximately 50% at 150 and 320 μE m⁻² s⁻¹, and 35-40% faster at 2050 μE m⁻² s⁻¹. This indicates that PetD strain is able to flux more electrons in through the cyt b6f complex than normally would be possible and therefore retain electron flux. These findings confirm that in the engineered PetD strain the electrons were channeled more effectively through the photosynthetic electron transport chain. In some embodiments, effective electron channeling leads to improved photosynthetic efficiency which can be translated into higher growth rate.

In some aspects, the increased photosynthetic efficiency results in increased cell growth and/or biomass production. In certain embodiments, increased biomass production encompasses increase production of biofuel, a biofertilizer, a nutraceutical, and/or a pharmaceutical. In particular embodiments, the methods increase carotenoid production in the engineered photosynthetic bacteria or plant compared to the native counterpart.

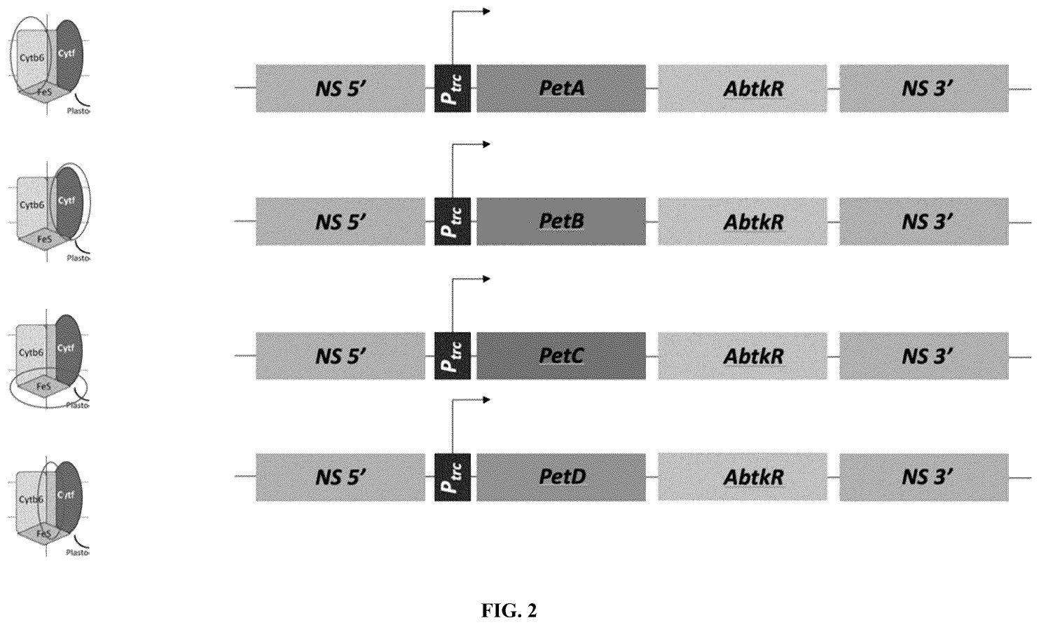

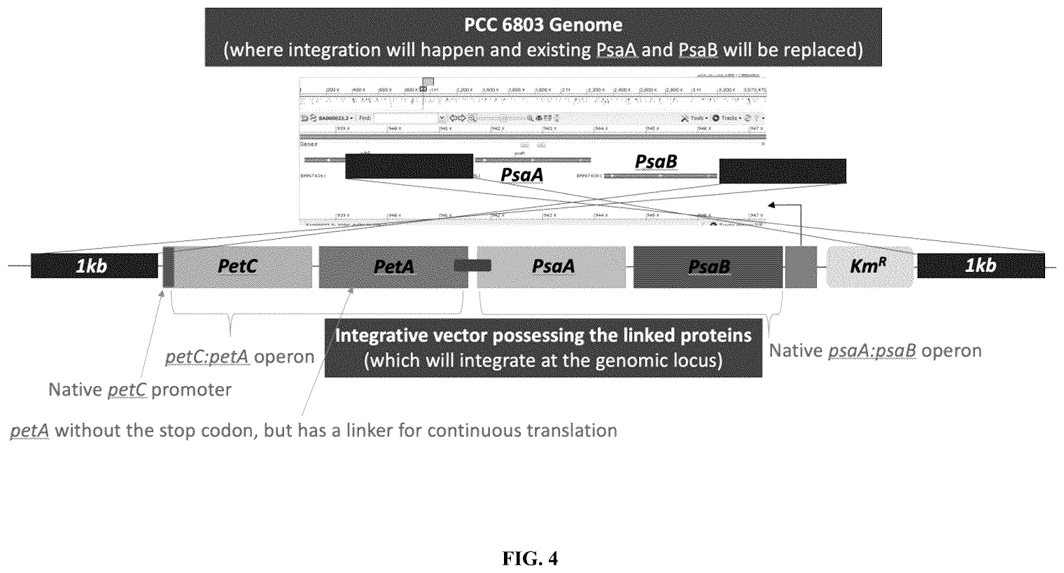

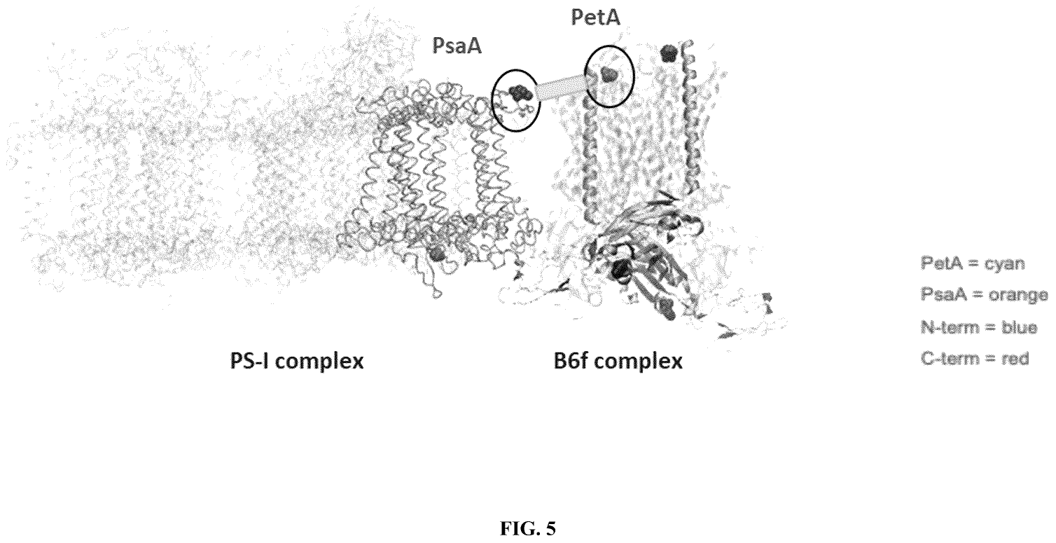

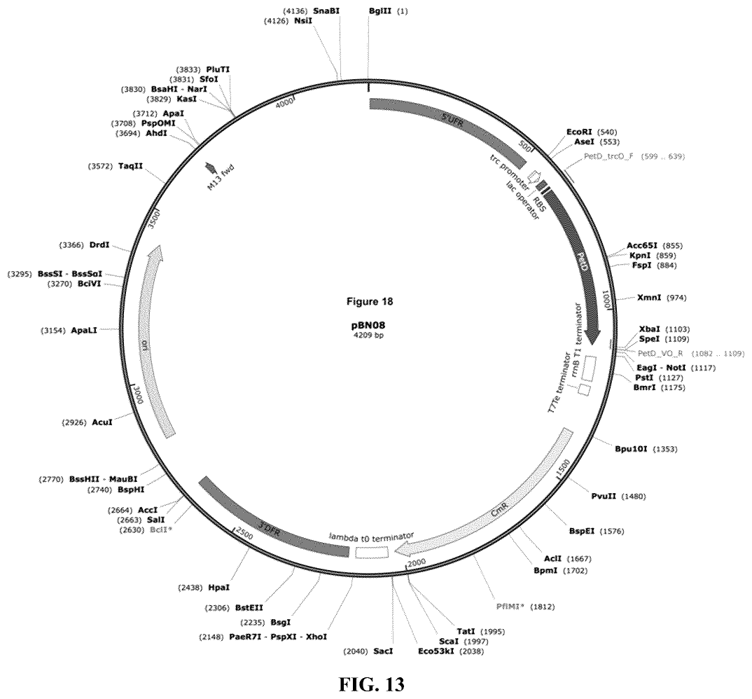

FIGS. 5A and 5B depict schematics illustrating the two strategies for engineering the photosynthetic electron transports. FIG. 2 depicts, in another exemplary implementation, the genetic constructs for overexpression of Pet subunits that are under the control of Ptrc promoter. FIG. 13 depicts, in another exemplary implementation, the genomic arrangements of cyt b₆f and photosystem protein for physically linking the two system. FIG. 4 depicts an exemplary protein linking strategy.

In some aspect, the method of increasing biomanufacturing by photosynthetic bacteria or a plant cell comprises engineering the photosynthetic bacteria or plant to overexpress a cyt b₆f major protein. In some implementations, the method comprises transforming the photosynthetic bacteria or a cell of the plant to express the cyt b₆f major protein using an overexpression promoter. In some embodiments, the overexpression promoter is an engineered promoter. In some embodiments, the overexpression promoter is a promoter from a naturally-expressed protein. In some embodiments, the overexpression promoter is selected from P_{cpc}, P_{rbc}, P_{psbA}, P_{psbA2}, P_{trc}, P_{lac}, P_{tac}, Pₙₚₜ, Pₕₛₚ₇₀ₐ-P_{rbcs2}, P_{petc}, and P_{SAD}. In some aspects, the overexpression promoter is a Pepe promoter (“cpc promoter”). In some aspects, the overexpression promoter is a P_{rbc} promoter (“rbc promoter”). In some aspects, the overexpression promoter is a P_{psbA} promoter (“psbA promoter”). In some aspects, the overexpression promoter is a P_{psbA2} promoter (“psbA2 promoter”). In some aspects, the overexpression promoter is a P_{lac} promoter (“lac promoter”). In some aspects, the overexpression promoter is a P_{lac} promoter (“tac promoter”). In some aspects, the overexpression promoter is a Pₙₚₜ promoter (“npt promoter”). In some aspects, the overexpression promoter is a Hsp70A-RBcS2. In some aspects, the overexpression promoter is a P_{SAD} promoter (“SAD promoter”). In some aspects, the overexpression promoter is a P_{petc} (“petC promoter”). In some aspects, the overexpression promoter is a P_{trc} promoter (“trc promoter”).

In another aspects, the method comprises engineering the photosynthetic bacteria or plant to express a synthetic construct of a cyt b₆f major protein linked to a photosystem protein. In some implementations, the method comprises transforming the photosynthetic bacteria or the cell of the plant to express a plasmid expressing a cyt b₆f major protein linked to a photosystem protein, for example, a protein from Photosystem II. In some embodiments, the operon expressing the photosystem protein in the genome of the transformed cyanobacteria is replaced with a nucleotide sequence expressing the cyt b₆f major protein linked to the photosystem protein. Thus, the synthetic construct comprises a linker sequence.

In yet other aspects, the linker sequence provides a flexible linker such as a glycine/serine linker. For example, the synthetic construct comprise a linker sequence encoding a polyglycine linker or a polyglycine+serine linker. In some embodiments, the linker sequence encodes linker having an amino acid sequence set forth in SEQ ID NO. 35, SEQ ID NO. 41, SEQ ID NO. 42, and SEQ ID NO. 43. In certain implementation, the linker sequence has a nucleic acid sequence set forth in SEQ ID NO. 34. In some aspects, it is important to clone the linker along with the nucleotide sequences of the peptides or proteins to be combined together. An exemplary method is to use PCR to amplify the proteins to be combined with the overhangs having a portion of the linker sequence. These overhang-possessing PCR amplicons can then be subjected to overlap extension PCR, Gibson assembly, or any similar method, whereby making these overhangs overlap and getting the uninterrupted and complete linker sequence.

In some aspects, the synthetic construct comprises a cyt b₆f major protein linked to a protein from Photosystem I. In certain embodiments, the plasmid comprises a first nucleotide sequence encoding petC:petA operon, a second nucleotide sequence encoding a linker sequence, and a third nucleotide sequence encoding a psaA-psaB operon. In some such embodiments, the plasmid encodes a contiguous translation of any one of PetC, PetA, PsaA, PsaB, and/or a combination thereof. In some aspects, the petC:petA operon lacks the sequence encoding a stop codon of petA. For example, the stop codon is replaced by the second nucleotide sequence of the plasmid thus resulting in the expression of petA linked to psaA. In some embodiments, the second nucleotide sequence comprises the sequence set forth in SEQ ID NO. 34.

In some aspects, the first nucleotide sequence encoding petC:petA operon, second nucleotide sequence encoding a linker sequence, and third nucleotide sequence encoding a psaA-psaB operon are codon sequence optimized for expression in the photosynthetic bacteria or plant being genetically engineered.

In certain embodiments, the plasmid further comprises a fifth nucleotide sequence and a sixth nucleotide sequence that are complementary to sequences flanking either and/or both of the 5′ and 3′ termini of the psaA-psaB operon of the cyanobacteria genome. In some aspects, the fifth nucleotide sequence and the sixth nucleotide sequences are each about 1 kb and are complementary to about a 1 kb sequence flanking either and/or both of the 5′ and 3′ termini of the psaA-psaB operon of a photosynthetic bacteria genome or of a plant cell genome.

Accordingly, engineered photosynthetic cells or organisms are described herein, wherein the engineered cell or organism overexpresses a cyt b₆f major protein or the engineered cell or organism expresses a synthetic construct of cyt b₆f major protein linked to a photosystem protein. In some aspects, overexpression of the cyt b₆f major protein (for example, PetD) is achieved by placing at least a portion of the gene expression of the cyt b₆f major protein in the engineered cell or organism under the regulation of a trc promoter (P_{trc}). In some aspects, the synthetic construct of cyt b₆f major protein linked to a photosystem protein comprises the cyt b₆f major protein linked to the protein from Photosystem I and is expressed via a modified psaA-psaB operon region. In some embodiments, the modified psaA-psaB operon region comprises a first nucleotide sequence encoding petC:petA operon; a second nucleotide sequence encoding a linker sequence; and a third nucleotide sequence encoding a psaA-psaB operon. In certain embodiments, the modified psaA-psaB operon region further comprises a fourth sequence encoding a petC promoter, and the petC promoter controls transcription of petC, petA, psaA, and psaB. In some implementations, the engineered organism is a cyanobacterium, for example one belong to the Synechocystis genus.

In a particular implementation, the existing natural psaA-psaB operon is from cyanobacteria and is replaced with the synthetic construct possessing linked petA:psaA under the control of natural promoter of petC (P_{petc}) (see FIG. 4). In such embodiments, this not only keeps the natural operons intact but also helps in reducing the intermolecular distance, ultimately improving the photosynthetic efficiency and biomass productivity. In some implementations, the method comprises transforming cyanobacteria with a plasmid comprising a first nucleotide sequence encoding petC:petA operon, a second nucleotide sequence encoding a linker sequence, and a third nucleotide sequence encoding psaA-psaB operon in which the plasmid encodes contiguous translation of PetC, PetA, PsaA, and PsaB. In some embodiments, the petC:petA operon encoded by the first nucleotide sequence lacks the sequence encoding the stop codon of petA, which is replaced by the second nucleotide sequence thus resulting in the expression of petA linked to psaA. In some aspects, the plasmid further comprises a fourth sequence encoding the petC promoter, which controls transcription of petC, petA, psaA, and psaB. In particular embodiments, the plasmid construct is (upstream) PetC/A promoter::PetC:PetA (linked with the linker sequence to) PsaA:PsaB::KanR (downstream). In some implementations, the plasmid comprises a fifth nucleotide sequence and a sixth nucleotide sequence, which are complementary to sequences flanking the psaA-psaB operon. In some aspects, the fifth nucleotide sequence and the sixth nucleotide sequence are 1 kb sequences that are complementary to a 1 kb region of cyanobacteria's genome flanking the psaA-psaB operon.

Cyanobacteria can naturally synthesize number of commercially viable compounds in response to environmental stimuli like dynamic light, UV radiations, etc. However, these capabilities can be commercially exploited, only upon demonstrating their outdoor scale-up cultivation. The engineered cyanobacteria strains disclosed herein can exhibit improved photosynthetic efficiency and hence improved biomass under variable light intensities. As such, their outdoor cultivation could be accelerated at scales feasible for commercial demonstration as well as implementation.

While photosynthetic light harvesting and carbon fixation is shared among all the oxygenic photoautotrophs, it is advantageous to perform the phototrophic engineering in cyanobacteria (e.g., Synechocystis and Synechococcus) due to its faster growth. Exemplary cyanobacteria strains include, but are not limited to, Synechocystis sp. PCC 6803, Synechococcus sp. PCC 7002, Synechococcus elongatus PCC 7942, Synechococcus sp. PCC 11901, and Synechococcus elongatus UTEX 2973. The exemplary embodiments in the present disclosure used cyanobacterial strain Synechocystis sp. PCC 6803 to simulate and identify the potential protein candidates to be linked or to overproduce. Though owing to similar principle of photosynthetic electron transport in prokaryotic and eukaryotic photoautotrophs, engineering strategies implemented and tested in Synechocystis can be effectively extended to plants, for example rice or corn varieties, thereby improving their yields and productivities.

In addition to the specific methods described in the Examples, the method of engineering the photosynthetic electron transport chain can also be implemented through markerless selection techniques. In some embodiments, the markerless selection technique is the clustered regularly interspaced short palindromic repeats (CRISPR) system. Genetically modified organisms developed through this approach, where the cell's natural architecture and components themselves have been used for improving its potential and quality, sometimes referred as ‘refactoring’, can be easily accepted in the commercial market. Other strategies for engineering the photosynthetic electron transport chain include the use of homologous recombination with counter-selection systems as well as self-excising cassettes (for example, Cre/lox or Flp/FRT systems).

A method of increasing the thylakoid membrane density of b₆f complex major proteins is also disclosed. In some embodiments, the method comprises overexpressing a cyt b₆f major protein. In some aspects, overexpression of the cyt b₆f major protein is through transformation of cyanobacteria with a plasmid comprising a nucleotide sequence encoding the cyt b₆f major protein under the control of an overexpression promoter, for example, the trc promoter (P_{trc}). Accordingly, the plasmid comprises an overexpression construct comprising a nucleotide sequence encoding the overexpression promoter and a nucleotide sequence encoding the cyt b₆f major protein. Transformation of cyanobacteria may result in integration of the overexpression constructs into the genome. In some aspects, the overexpression constructs are integrated into the genome of the cyanobacteria at the neutral locus. In some implementations, the plasmids comprising the overexpression constructs further comprise a chloramphenicol resistance cassette. In particular implementations, the method of increasing the thylakoid membrane density of b₆f complex major proteins comprises overexpressing PetD. In some aspects, the plasmid is constructed from the PEERM4 vector backbone (for example, Addgene ID #64025 or 64026), though with the nrsB promoter replaced by the tre promoter. In a particular implementation, the plasmids for overexpressing a cyt b₆f major protein is selected from pBN05, pBN06, pBN07, and pBN08, the sequences of which are set forth in SEQ ID NOs. 36-39.

In certain implementation of increasing the thylakoid membrane density of b₆f complex major proteins in cyanobacteria, plasmid based on the pUC vector may be used, especially if the overexpression of the cyt b₆f major protein is achieved through homologous recombination. In other implementation, overexpression of a protein in cyanobacteria may be achieved using RSF1010 replicative plasmids.

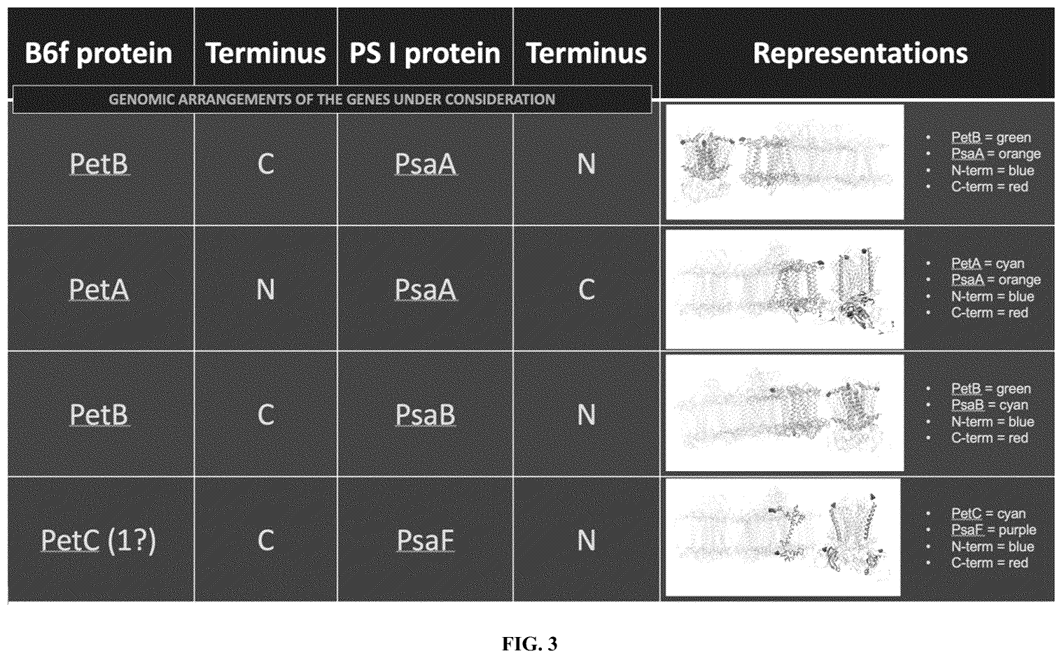

In some embodiments, the method of linking the photosynthetic complexes using covalent peptide linkers comprises transforming a cyanobacteria with a plasmid expressing a cyt b₆f major protein linked to a photosystem protein. In some such embodiments, the operon expressing the photosystem protein in the genome of the transformed cyanobacteria is replaced with a nucleotide sequence expressing the cyt b₆f major protein linked to the photosystem protein. In one aspect, the cyt b₆f is linked to Photosystem I, for example, the linkage between cyt b₆f and Photosystem I is as shown in FIG. 3. In another aspect, the cyt b₆f is linked to Photosystem II. In yet another aspect, the cyt b₆f is linked to Photosystem I and Photosystem II. The linker sequence is designed such that the structure of adjoining functional proteins remains unaffected. Thus, the linker sequence prefers provides a flexible linker such as a glycine/serine linker. In certain embodiments, the linker sequence encodes a polyglycine linker or a polyglycine+serine linker. In some embodiments, the linker sequence comprises the sequence set forth in SEQ ID NO. 34 or encode an amino acid sequence comprising the sequence set forth in SEQ ID NO. 35, SEQ ID NO. 41, SEQ ID NO. 42, or SEQ ID NO. 43.

The disclosed methods of increasing photosynthetic efficiency provide advantages over existing technology for biomanufacturing and increased sustainability. Cyanobacteria are considered as phototrophic cell factories for metabolic engineering. However, these engineering implementations, especially for overproduction of natural or heterologous chemicals from the system, tend to reduce the central carbon flux towards the biomass. Therefore, wildtype strains grow better than the engineered strains. Under these circumstances, using photosynthetically efficient model strains as the basis for overproduction of natural or heterologous chemicals could be advantageous using the wild type strains of cyanobacteria. Furthermore, while growth performances of cyanobacteria are not competitive with already existing heterotrophic platforms, engineering cyanobacteria strains which are already exhibiting improved growth performances under variable light intensities, would be beneficial. Accordingly, methods of increasing biomanufacturing are disclosed comprising engineering photosynthetic bacteria or plants to overexpress a cyt b₆f major protein or to express a synthetic construct possessing linked petA:psaA under the control of natural promoter of petC (P_{petc}) prior to modifying the photosynthetic bacteria or plant for producing a biofuel, abiofertilizer, food, a nutraceutical, and a pharmaceutical.

### EXAMPLES

The present disclosure is further illustrated by the following examples that should not be construed as limiting. The contents of all references, patents, and published patent applications cited throughout this application, as well as the Figures, are incorporated herein by reference in their entirety for all purposes.

### Example 1. Introduction of Covalent Linkers Between b6f and PSI to Avoid Electron Leakage Via a Mobile Electron Transporter Plastocyanin (PC)

### 1. Computational Screening of Potential Proteins to be Linked

PSI is a multi-protein complex, having PsaA, PsaF and PsaB as the major proteins, whereas cyt b₆f is made up of PetA, PetB, PetC and PetD major proteins. The mobile electron carriers like PC, carry electrons from cyt b₆f to PSI. Therefore, getting these two complexes together could result in improved photosynthetic efficiency. Computational 3D protein analysis was performed to identify suitable proteins to link cyt b₆f and PSI. Based on the peptide proximities and their N-C termini orientation in the thylakoid membrane of Synechocystis, four different peptide combinations were selected: a) PetB_C-term: PsaA_N-term, b) PetA_N-term: PsaA_C-term, c) PetB_C-term: PsaB_N-term, and d) PetC_C-term: PsaF_N-term, using the computational models. Considering their gene arrangements in the genomic operons and intermolecular distance, PetA_N-term: PsaA_C-term combination was selected for linking.

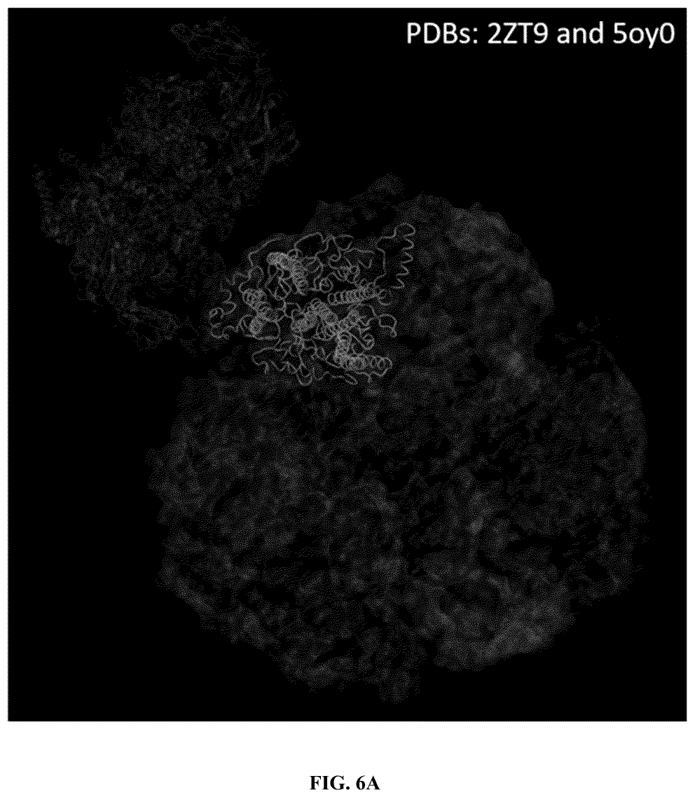

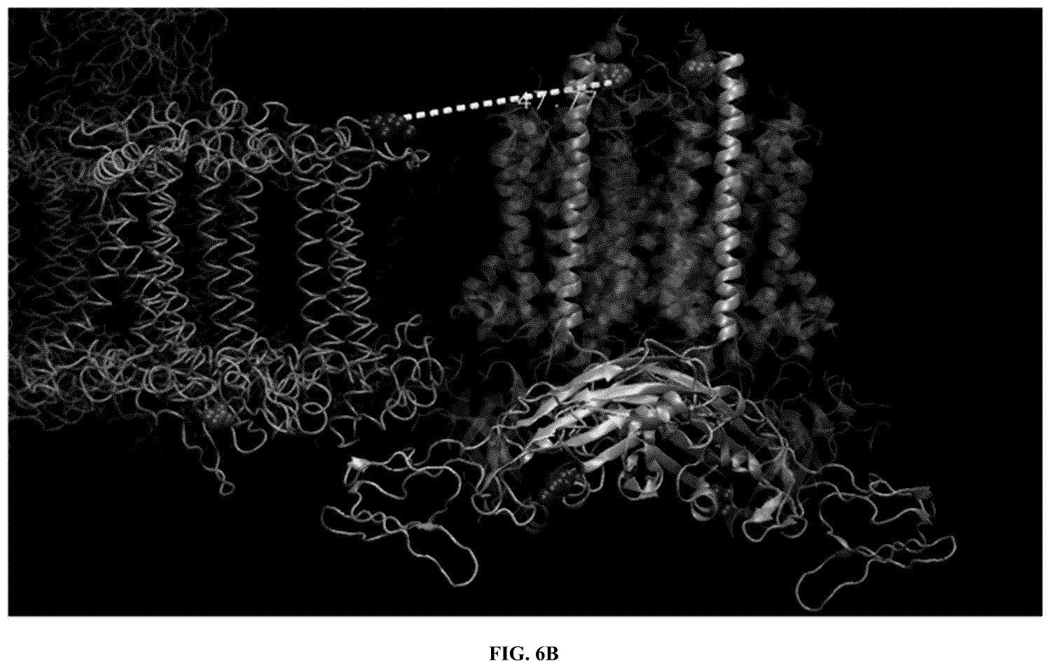

A computational modeling of cyanobacterial super-complex analysis was conducted consisting of Photosystem I (PSI) and the cytochrome b₆f complex based off a microalgae Chlamydomonas reinhardtii structure from Steinbeck et al. The plan was to fuse the core subunits for both complexes and measure the distance between N and C termini. Selection was narrowed down to only the core subunits in which the N and C termini are on the same side of the membrane and have the shortest distance from one another. The two subunits chosen were PsaA from PSI and PetA from cytochrome b6f. The results of the computational modeling of cyanobacterial super-complex are shown in FIG. 6.

### 2. Synthesis of Covalently Linked Cyt b₆f and PSI in Synechocystis

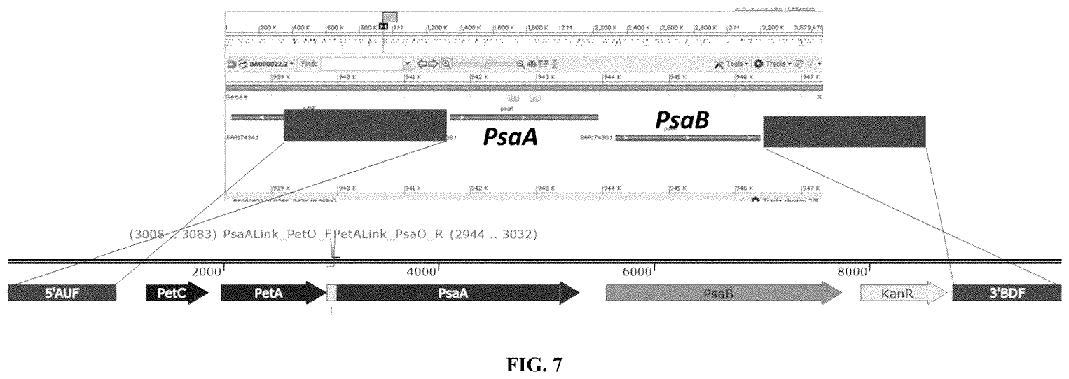

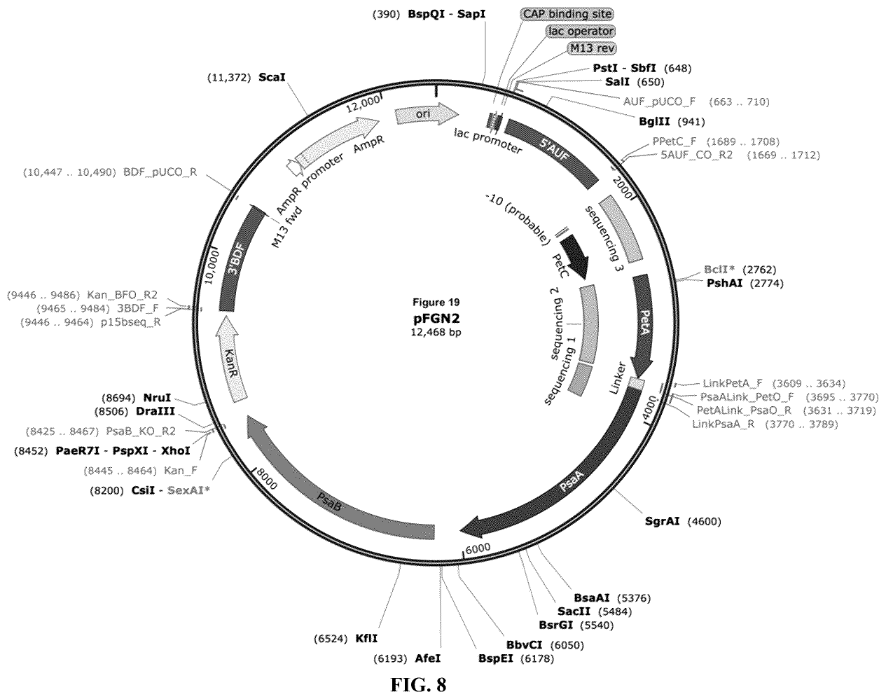

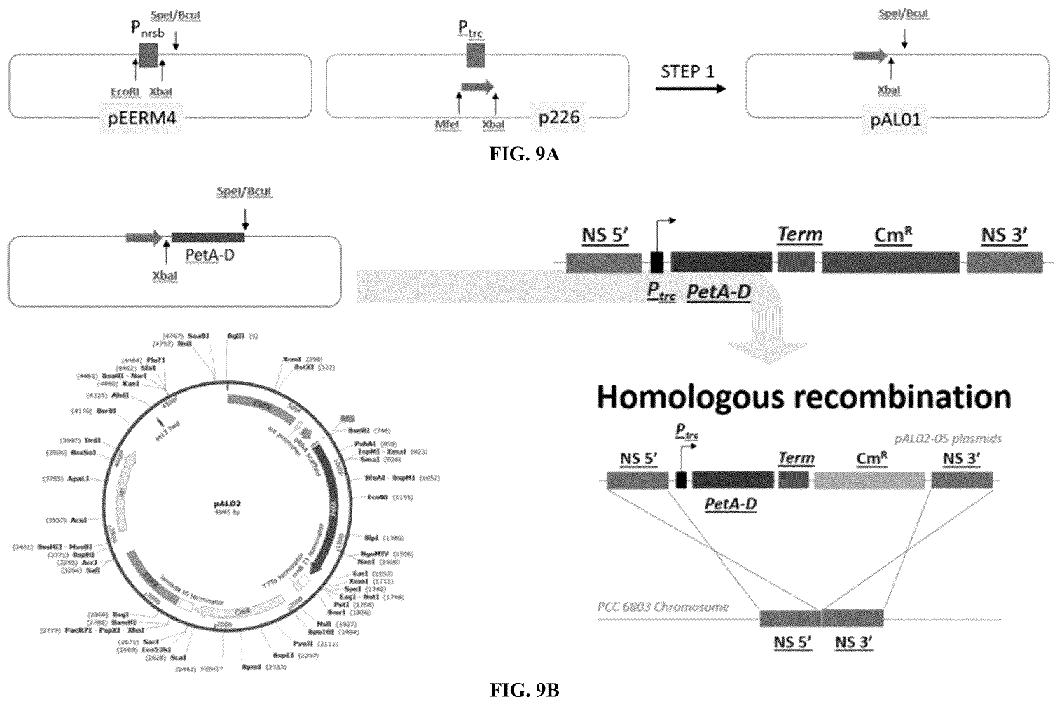

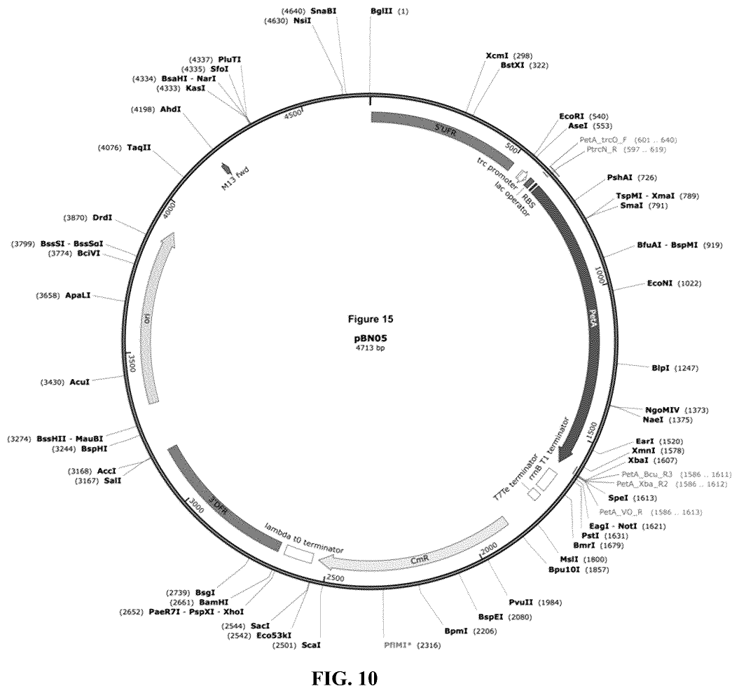

A plasmid-based construct was made to, a) link PetA and PsaA peptides together with a synthetic linker sequence, and b) replacing the naturally existing genomic region of psaA:psaB operon by this synthetic linker construct, through homologous recombination strategy (FIG. 7). As petC:petA and psaA:psaB form an operon within the genome of Synechocystis, while designing the plasmid, it was ensured to keep these operonic genes together. Therefore, as depicted in the figure, the entire big cargo of petC:petA-linker-psaA:psaB was regulated by upstream sequence of petC gene. The linker peptide (sequence in the figure) was introduced by replacing the stop codon from petA and start codon from psaA, ensuring their continuous translation as PetA-Linker-PsaA from the RNA transcript. Secondly, as this cargo along with the kanamycin resistance cassette was integrated into the genome by replacing naturally existing copy of psaA:psaB operon, upon complete integration, the engineered Synechocystis will have only one copy of these two genes, only constituted by the linked operon construct. The linker strain was created by transforming Synechocystis with pFGN2 plasmid, the sequence of which is set forth in SEQ ID NO. 40. FIG. 8 depicts the map of the pFGN2 plasmid.

Synechocystis strains engineered to have closer cyt b₆f and PSI are referred to herein as “Linker strains.”

### Example 2. Enhancing the Density of the Redox Complexes in the Thylakoid Membrane

Unlike the strategy of Example 1, which involves covalently linking the two photosynthetic complexes together, this strategy involved increasing the inter-complex protein density, ultimately reducing the physical distance between them. For its implementation, four major cyt b₆f proteins, PetA, PetB, PetC, and PetD were selected as the potential candidates for overexpression, as cyt b₆f is architecturally between PSII and PSI. Strains were constructed to overexpress the major proteins PetA, PetB, PetC, and PetD with the assumption that overexpressing one of the major proteins would result in increasing the density of cyt b6f by leveraging unknown endogenous regulatory systems.

Synechocystis strains were engineered for their individual overexpression under the control of Pure promoter. Like previous strategy, these constructs were integrated into the genome, however at the widely used neutral locus within the genome. The plasmids were constructed as given in the figure along with chloramphenicol resistance cassette.

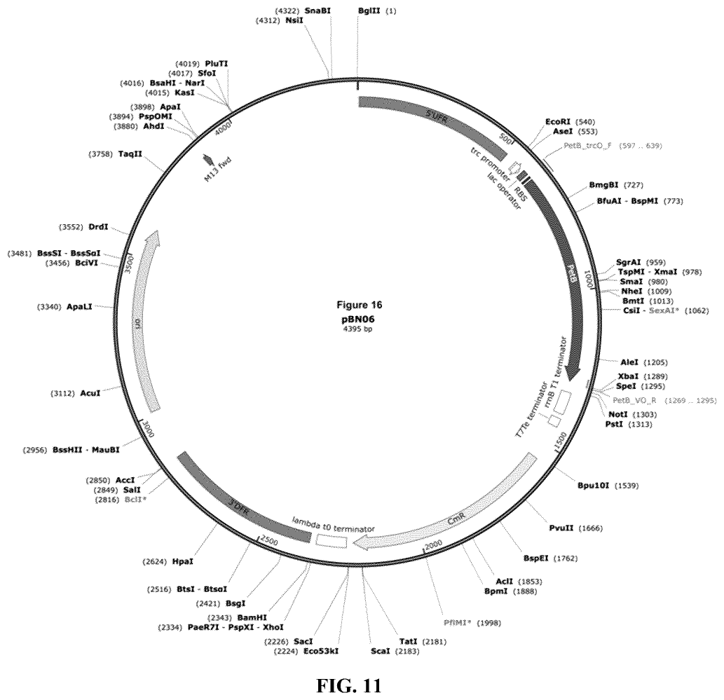

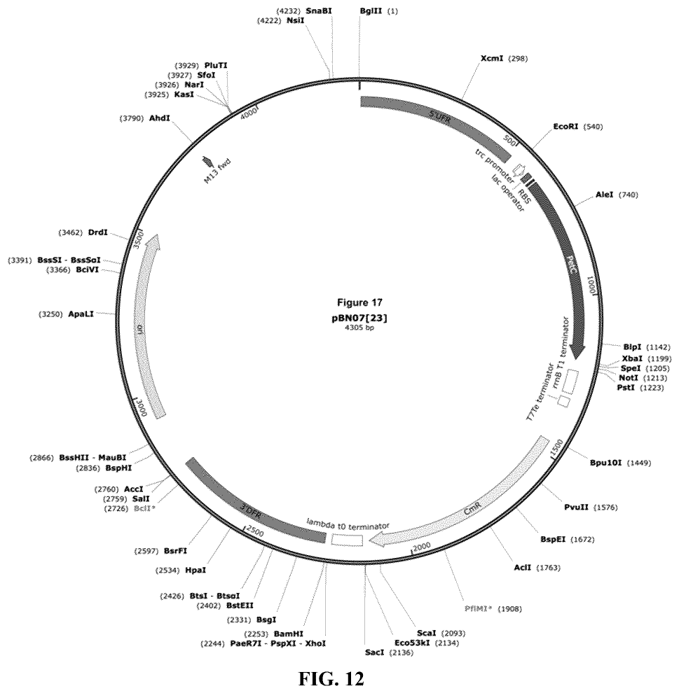

Synechocystis strains were engineered for their individual overexpression under the control of Ptre promoter. Like previous strategy, these constructs were integrated into the genome, however at the widely used neutral locus within the genome. The plasmids were constructed as given in the figure along with chloramphenicol resistance cassette. PetA overexpression used pBN05, the map of which is shown in FIG. 10. PetB overexpression used pBN06, the map of which is shown in FIG. 11. PetC overexpression used pBN07, the map of which is shown in FIG. 12. PetD overexpression used pBN08, the map of which is shown in FIG. 13.

### Example 3. Estimating the Effect of Both the Strategies on the Cell Growth and Photosynthetic Efficiency

Engineered strains produced from the approaches of Example 1 and Example 3 were examined for their growth rate (based on spectrophotometrically determined culture density or OD₇₃₀) and photosynthetic efficiency (based on oxygen evolution study) in comparison to the WT Synechocystis.

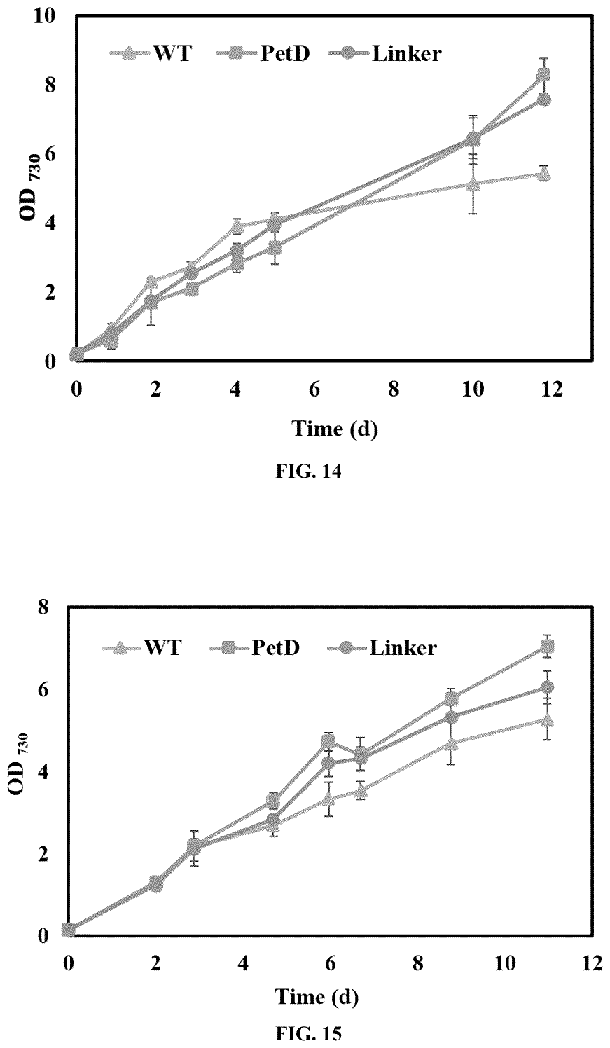

Constructed strains are cultured in various light intensities of low (20 μmol of photons m⁻²s⁻¹), moderate (80 μmol of photons m⁻²s⁻¹), and high (150 μmol of photons m⁻²s⁻¹) as well as various CO₂ concentration of atmospheric (0.04%), and 1%. Growth of PetD, Linker and wild-type PCC 6803 (control) strains in shake flasks at 250 rpm and under atmospheric CO₂ and 20 μmol of photons m⁻²s⁻¹ is shown in FIG. 14. Under this condition, 52% and 39% increase in cell density of the strain expressing PetD and Linker compared to the WT at day 12 was observed, respectively. Also, Growth of PetD, Linker and wild-type PCC 6803 (control) strains in shake flasks at 250 rpm, under atmospheric CO₂ and 80 μmol of photons m⁻²s⁻¹ is shown in FIG. 15. The experimental comparison test resulted in 34% and 15% increase in cell density of the strain expressing PetD and Linker compared to the WT at day 11, respectively. These findings confirm our hypothesis that in the engineered PetD strain the electrons were channeled more effectively through the photosynthetic electron transport chain and thereby, improved photosynthetic efficiency which can be translated into higher growth rate.

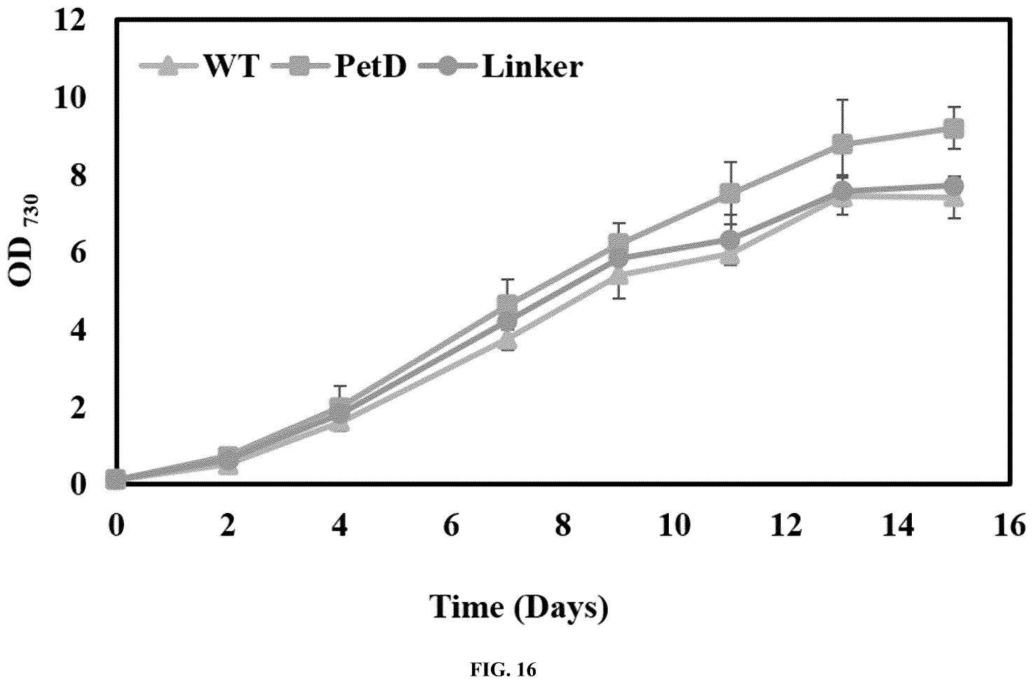

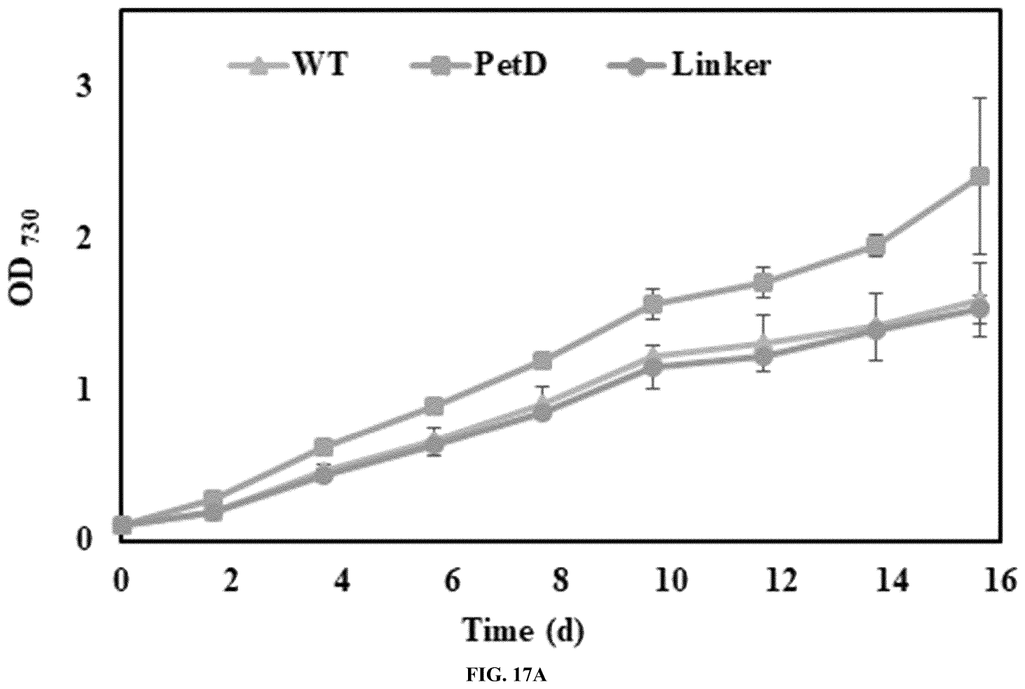

Also, growth of PetD, Linker and wild-type PCC 6803 (control) strains under 1% CO₂ and low light (80 μmol of photons m⁻²s⁻¹) is depicted in FIG. 16. Results showed a similar growth for wild-type and Linker while PetD showed % 24 increase in OD₇₃₀ compared to control at day 9.

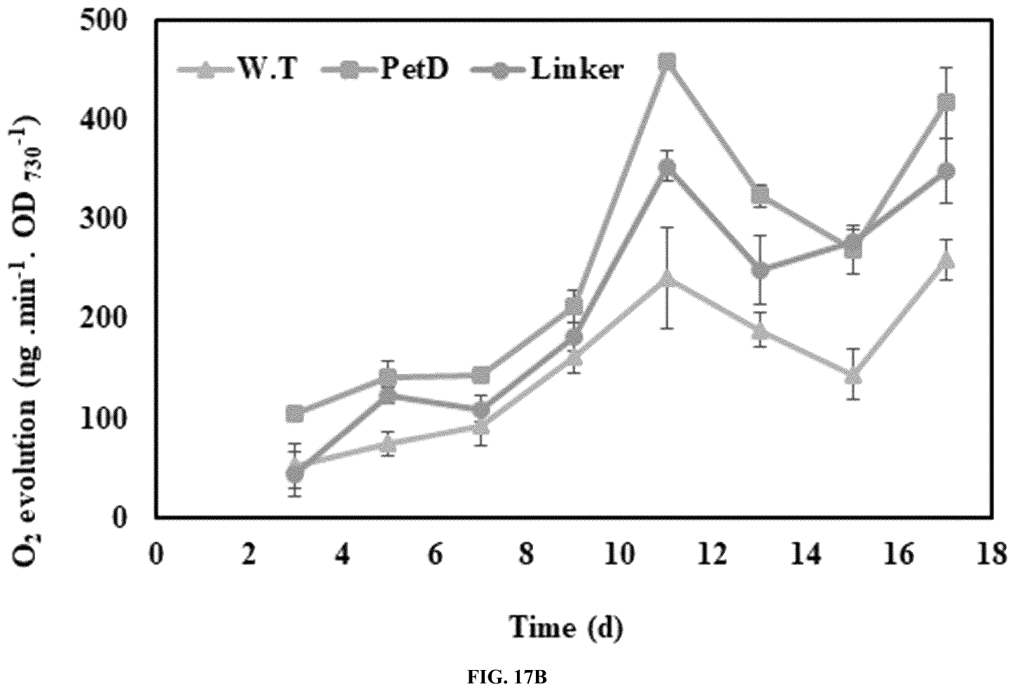

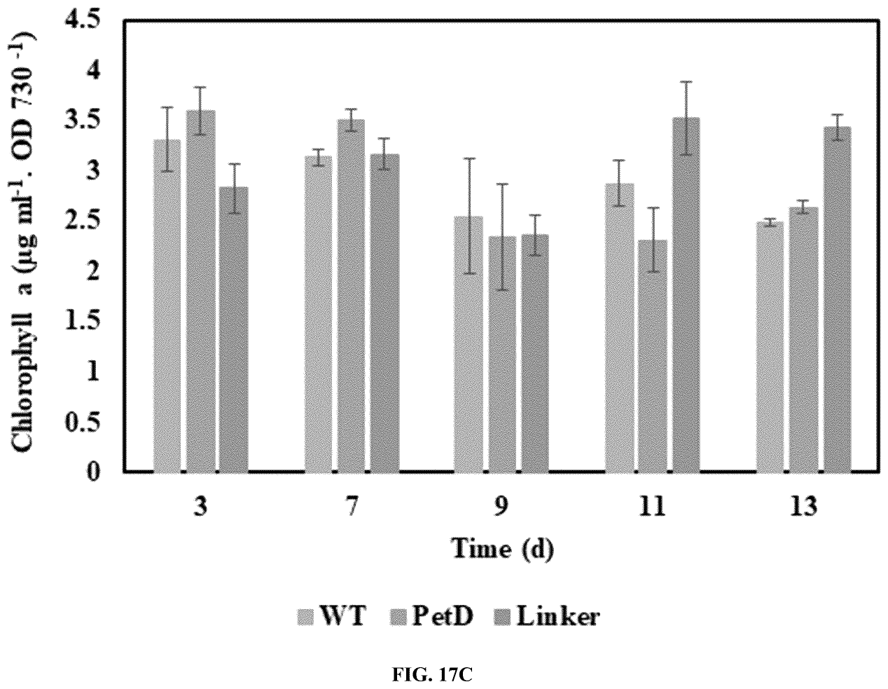

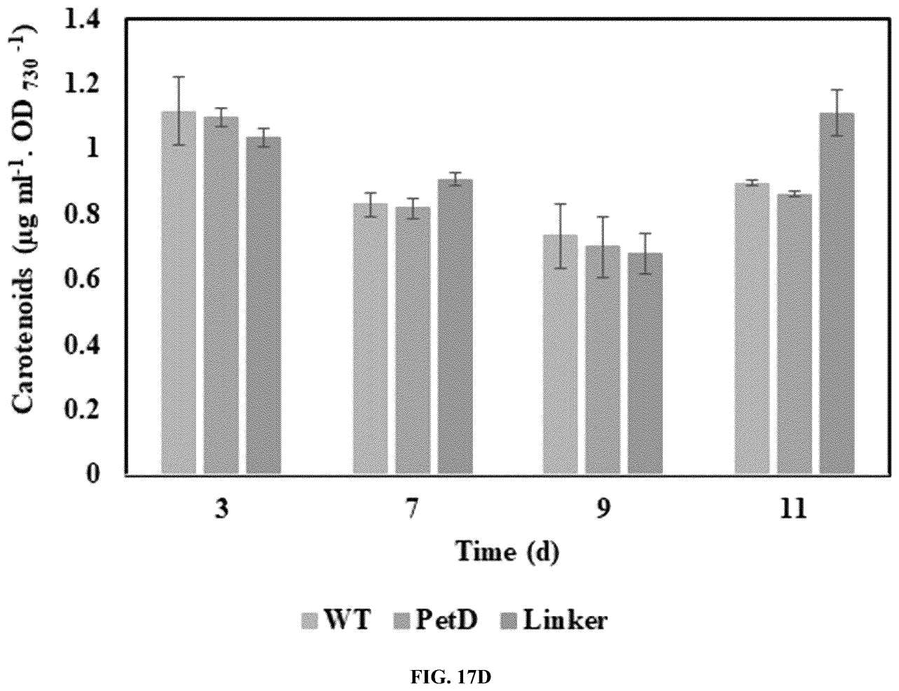

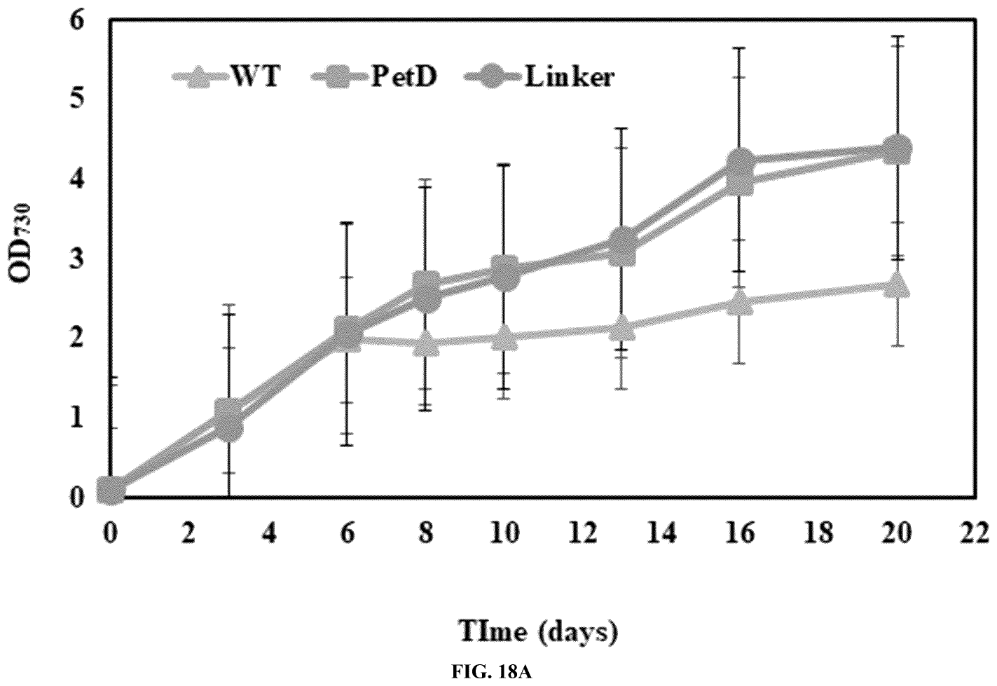

As cells are more exposed to light energy, more proton is produced which increases the electron transport rate in the cell. On the other hand, less light exposure results in lesser proton availability and hence, lesser NADPH production in the cells. In darker conditions, strains with a better electron transport can produce more of electron carrier NADPH that can be further used in Calvin cycle to fix CO₂. In this regard, further analysis was performed under darker condition to highlight the differences between the engineered strains and control in the term of NADPH and cell productivity. The experiment was performed in containers with larger diameter under atmospheric CO₂ to provide darker conditions and less exposure to the cells. Results are shown in FIGS. 17A-17D. As can be visible in FIG. 17A, PetD strain was showing a % 51.6 growth compared to the other two strains. This validates the higher electron transport efficiency in the engineered strains. Oxygen evolution study for this test was also showing higher photosynthetic efficiency of PetD and linker strains over wild-type 6803 which is shown in FIG. 17B. Further tests were performed to measure the chlorophyll-a and carotenoids content in the strains that are shown in FIGS. 17C and 17D, respectively. From FIG. 17C, PetD strain having slightly higher Chl-a compared to other strains in the initial phase of cultivation that is mainly indicative of the healthier cells. Numerous cyanobacteria, the photosynthetic microorganisms pivotal in shaping our planet through their groundbreaking oxygen production, utilize the Orange Carotenoid Protein (OCP) to safeguard their delicate photosystems from the detrimental impacts of excessively absorbed light that produces reactive oxygen species which are toxic to the cells. Hence, the carotenoid content of the cells was measured, and results show that PetD accumulated lesser amount of this pigment compared to wild-type and Linker which shows the lesser amount of reactive oxygen species in this engineered strain and better performance of photosystem for channeling electrons.

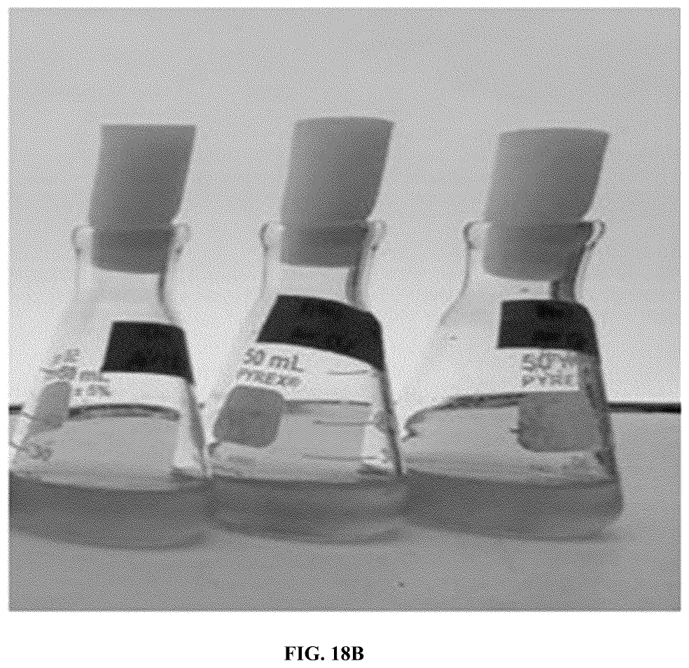

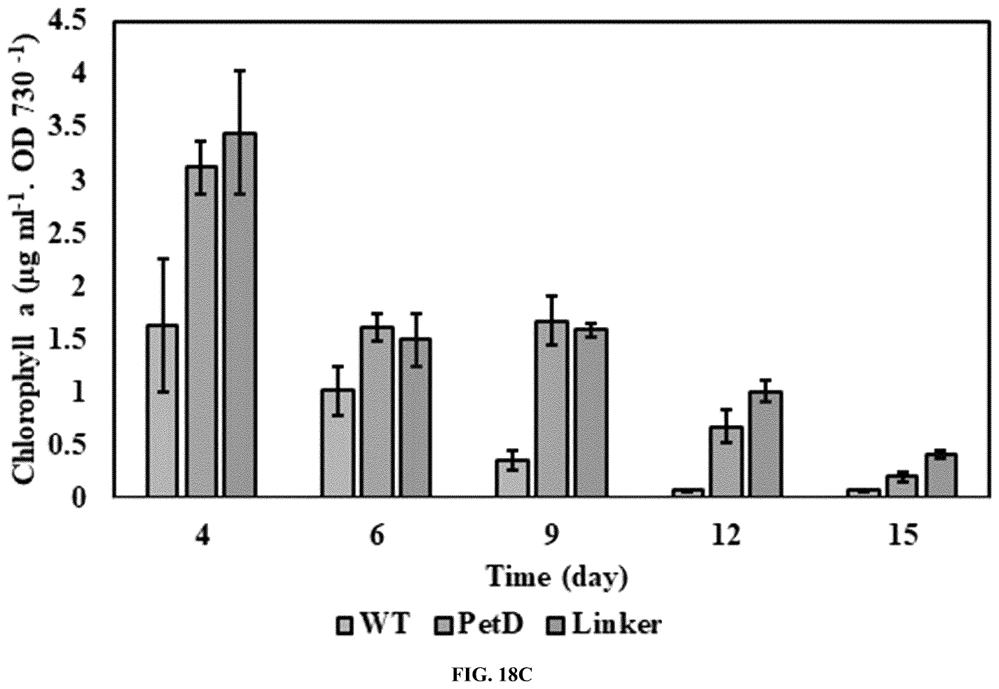

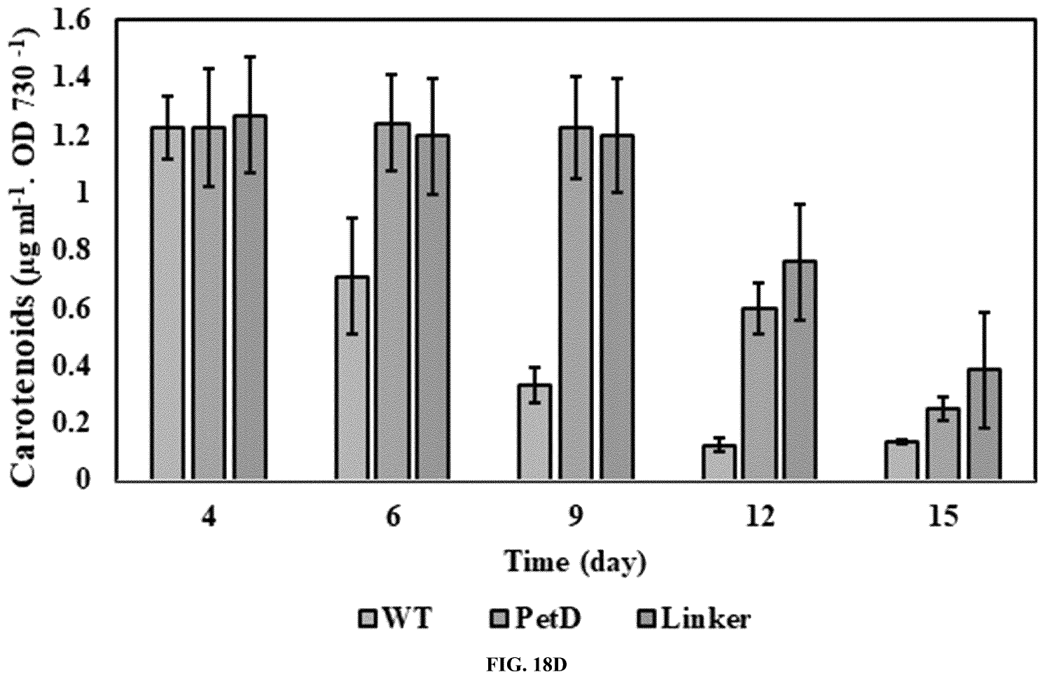

The correlation between photosynthetic rate and cell growth in cyanobacteria hinges on a nuanced balance between the generation of reductant and ATP and their utilization as metabolites, notably in CO₂ fixation and biomass formation. Under conditions of ample light, wherein CO₂ might pose a limiting factor, surplus light is dissipated through either heat or fluorescence emitted from the antennae. Hence, an experiment was performed in shake flasks under atmospheric CO₂ and high light of 150 μmol of photons m⁻²s⁻¹ to compare the growth efficiency and phenotypic changes between the engineered and wild-type strains. Results are shown in FIGS. 18A-18D. As can be visible in FIG. 18A, all strains were growing with a similar trend until day 6. After day 6, wild-type strains reached the stationary phase, while the other two engineered strains continued their growth until day 16 when they reached their stationary phase. What can be concluded from this experiment is that the main advantage of overexpression of PetD and linking PSI to cyt b6f is longer growth phase and hence, higher cell productivity under abundant light exposure. Interestingly, after 6 days, all strains reached the OD₇₃₀ of ˜2 under 150 μmol of photons m⁻²s⁻¹ while under 20 μmol of photons m⁻²s⁻¹ and 80 μmol of photons m⁻²s⁻¹ the OD730 of close to 4 obtained that shows the unpleasant condition of 150 μmol of photons m⁻²s⁻¹ for the cells. As cells turned yellow after 3 days of cultivation (shown in FIG. 18B), a chlorophyll a content measurement was performed which is depicted in FIG. 18C that shows the PetD and Linker had healthier photosystem compared to control. The carotenoid content result is also shown in FIG. 10D.

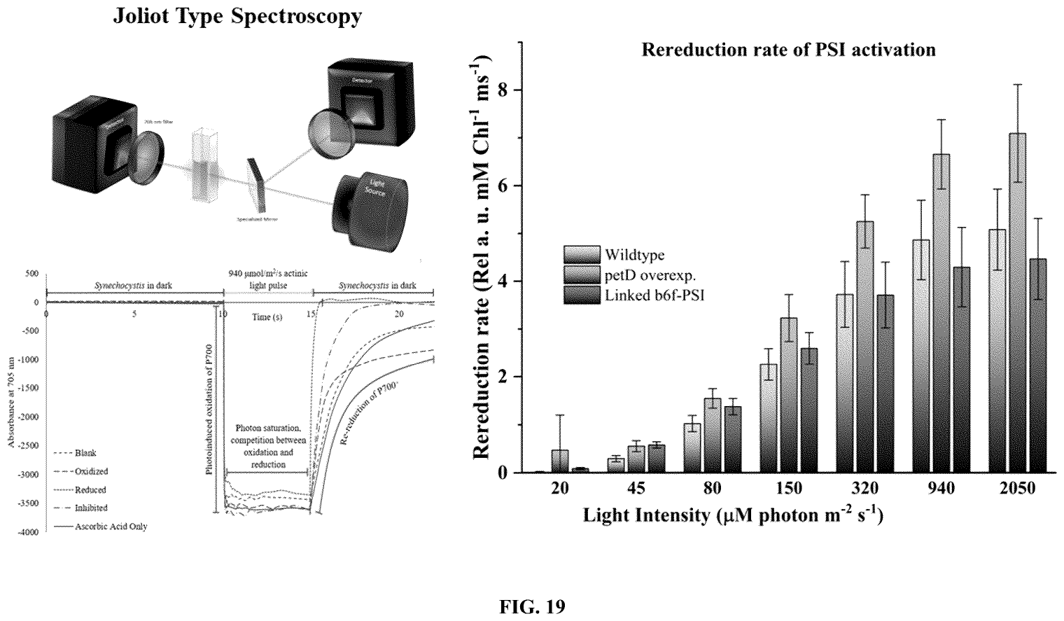

The Joliot type spectroscopy is a specialized type of pump-probe spectroscopy. It does so by first pumping a flash of actinic light and then probing the absorbance at 680 nm-which can provide a signal specifically for the rereduction of PSI's (P700⁺→P700) so that it may be able to accept a new photon. Each absorbance unit recorded can be considered a single PSI unit re-reducing P700⁺→P700 and therefore can be translated as the electron flow. This experiment involved comparing wild-type cells, PetD cells, and Linker b6f cells in triplicate studies. All three samples were tested for PSII and b6f inhibition using DCMU and DBMIB, respectively and the results reflected that all three samples similarly were inhibited by both DCMU and DBMIB, indicating that the functioning of both PSII and the cyt b₆f were main contributors to the electron flux. Looking at the main test results that is shown in FIG. 19 and beginning with lower light intensities it was immediately apparent the genetically engineered strains were rereducing faster than WT. The PetD cells rereduced 67 times faster than WT at 20 μmol of photons m⁻²s⁻¹. The PetD and linker rereduced at double the rate of wildtype at 45 μmol of photons m⁻²s⁻¹, As the light intensity increases the PetD cells maintained faster than that of WT, approximately 50% at 150 and 320 μmol of photons m⁻²s⁻¹, and 35-40% faster at 2050 μmol of photons m⁻²s⁻¹. This is interesting considering that higher light intensities initiate high dissipation due to photo inhibition and PSII is still intact and present in both PetD cells. This may indicate that PetD strain is able to flux more electrons in through the b6f complex than normally would be possible and therefore retain electron flux. The Linker cells were 11 times faster than the WT at 20 μmol of photons m⁻²s⁻¹ and were faster than all samples at 45 μmol of photons m⁻²s⁻¹, almost double that of WT. At 80 and 150 μmol of photons m⁻²s⁻¹, the Linker cells are still rereducing faster than WT, 35% and 15% faster. However, WT is faster than Linker cells at light intensities above 150 μmol of photons m⁻2s⁻¹.

### Example 4. Materials and Methods

### 1. Strains and Growth Conditions

All cloning was performed in the Escherichia coli strain DH5a. Cells were grown at 37° C. in LB media either in liquid or on agar plates supplemented with 25 μg/mL chloramphenicol or 20 μg/mL kanamycin as required.

A wild-type strain of Synechocystis 6803 (WT) and the recombinant strains of PetA, PetB, Pet C, PetD and Linker were grown at 30° C. in liquid or solid BG-11 medium. Chloramphenicol at a concentration of 10 μg/mL for Pet strains or Kanamycin at a concentration of 20 μg/mL for Linker strain were added to the BG-11 medium when required. Experiments that performed in ambient air were cultured in 50 ml Erlenmeyer flasks with a working volume of 10 ml and a speed of 250 rpm. While higher concentrations of CO₂ were needed to provide for the cells, cultures were grown in bubble cultures with a working volume of 50 ml. Experiments are designed to be conducted in low light (20 μmol of photons m⁻²s⁻¹), moderate (80 μmol of photons m⁻²s⁻¹) and high light (150 μmol of photons m⁻²s⁻¹) intensities. An Agilent Cary 60 UV-Vis Spectrophotometer was used to measure the OD₇₃₀ of the cultures in order to monitor the growth of the cells.

### 2. Plasmid Construction

Plasmids pBN05-08 were constructed from pEERM4 vector in which the nrsB promoter was replaced by trc promoter. Using this, the petA, B, C and D are expressed by the trc promoter. The linker recombinant, PUC19 vector was used as the backbone and the entire operon cloned to the vector using overlap extension PCR cloning (PetC upstream−PetC/A promoter::PetC:PetA (linked with a linker to) PsaA:PsaB::KanR-PetA downstream) that was supposed to express at PetC-PetA operon locus in the 6803 genome. Table 1. lists the oligonucleotides used in this study.

TABLE 1

| Primer name | Sequence | SEQ ID NO. |
|---|---|---|
| Ptrc_Eco_F | aactgaattcttgacaattaatcatccggc | 1 |
| Ptrc_Xba_R | ctggagatctagttaatttctcctctttaatg | 2 |
| PetA_Xba_F | ATTAtctagaATGAGAAACCCTGATAC | 3 |
| PetA_Spe_R | GCGTactagtCTAGAAATTAAGTTCGGC | 4 |
| PetB_Xba_F | CGGCtctagaATGTTTTCAAAAGAAGTC | 5 |
| PetB_Spe_R | GCGTactagtTTACAAAGGACCGGAAATG | 6 |
| PetC_Xba_F | CGGCtctagaATGTTAGTAAAAATCCTTAAAT | 7 |
| PetC_Spe_R | CGTGactagtCTAAGCCCACCAGGGAT | 8 |
| PetD_Xba_F | CGGGtctagaATGAGTATTATCAAAAAGC | 9 |
| PetD_Spe_R | CGTGactagtTTAGAACAAGCCCAAGG | 10 |
| EYFP_RBS_Xba_F | ATTTtctagaAGGAGATATAATAtggtgagcaagggcgaggag | 11 |
| EYFP_Spe_R | ACTGactagtttattacttgtacagctcgtc | 12 |
| PetA_VO_R | ATTATATCTCCTtctagaCTAGAAATTAAGTTCGGCAGC | 13 |
| PetA_trcO_F | TTCACACAGGAGATATAATATGAGAAACCCTGATAC | 14 |
| | TTTG | |
| PtrcN_R | ATTATATCTCCTGTGTGAAATTG | 15 |
| PtrcN_VO_F | cttcttgagatcctttttttgaattcTTGACAATTAATCATC | 16 |
| PetB_trcO_F | CAATTTCACACAGGAGATATAATATGTTTTCAAAAG | 17 |
| | AAGTCAC | |
| PetB_VO_R | ATTATATCTCCTtctagaTTACAAAGGACCGGAAATGC | 18 |
| PetC_trcO_F | CAATTTCACACAGGAGATATAATATGTTAGTAAAAA | 19 |
| | TCCTTAAA | |
| PetC_VO_R | ATTATATCTCCTtctagaCTAAGCCCACCAGGGATC | 20 |
| PetD_trcO_F | ATTTCACACAGGAGATATAATATGAGTATTATCAAA | 21 |
| | AAGCC | |
| PetD_VO_R | ATTATATCTCCTtctagaTTAGAACAAGCCCAAGGTTAG | 22 |
| PetA_Xba_R2 | TTCAtctagaCTAGAAATTAAGTTCGGCAGC | 23 |
| PtrcN_Eco_F2 | GAACgaattcTTGACAATTAATCATC | 24 |
| PetD_Xba_R2 | ATTCtctagaTTAGAACAAGCCCAAGGTTAG | 25 |
| PetA_F | GACAGATAGTGGGCTAAGAATAAC | 26 |
| PetA_R | CGGTACAACTTAGCCACC | 27 |
| PetB_F | CTTTGCTCAAACCATTTGG | 28 |
| PetB_R | CCACCAACTCCTTGAGAATG | 29 |
| PetC_F | GATAGTGGGCTAAGAATAACTTTGC | 30 |
| PetC_R | CCAACTCCTTGAGAATGACC | 31 |
| PetD_F | CCTTAATAGTTCATTGTCGAGCG | 32 |
| PetD_R | GACAAGCTTAATTACCTAACAGAGG | 33 |

An exemplary linker has the amino acid sequence of PTLGTSGGGGGSGGGGGSGGGGGSTSGIDLEP (SEQ ID NO. 35). In the linker recombinant of this example, the nucleic acid sequence encoding this linker has the sequence of CCTACCTTAGGGACTAGTGGAGGCGGAGGGGGCAGCGGAGGCGGAGGGGGGAGTG GGGGCGGCGGCGGTAGCACTAGTGGAATTGACCTGGAACCC (SEQ ID NO. 34). Other linkers have the amino acid sequence of: PTLGM(GGGGGS)DLEP (SEQ ID NO: 41), PTLGM(GGGGGS/GGGGGS)DLEP (SEQ ID NO: 42), PTLGM(GGGGGS/GGGGGS/GGGGGS)DLEP (SEQ ID NO: 43). For designing the nucleic acid sequence encoding these linkers, codon optimization strategies may be utilized. In some aspects, it is important to clone the linker along with the nucleotide sequences of the peptides or proteins to be combined together. For example, PCR is used to amplify the proteins to be combined with the overhangs having ‘partial’ linker sequences. These overhang possessing PCR amplicons can then be subjected to overlap extension PCR or Gibson assembly or any similar method, whereby making these overhangs overlap and getting the uninterrupted and complete linker sequence.

The sequences of the plasmids are provide as follows:
a. pBN05: SEQ ID NO. 36
b. pBN06: SEQ ID NO. 37
c. pBN07: SEQ ID NO. 38
d. pBN08: SEQ ID NO. 39
e. pFGN2: SEQ ID NO. 40

### 3. Construction of Cyanobacterial Strains

Natural transformation was performed for all the PetA, PetB, PetC, PetD and Linker strains. Wild-type PCC 6803 was initially cultivated on BG11 agar plates and then transferred to 15 ml Erlenmeyer flasks. When the OD730 reached to 0.5, plasmids were transformed to wild-type cells and incubated under 50 μmol of photons m⁻²s⁻¹ overnight and then streaked over the BG11 plates containing antibiotics. After the appearance of colonies, they were segregated by multiple rounds of culturing on the BG11 agar plates containing antibiotics corresponding antibiotics. Colony PCR performed to confirm the availability of constructs in the genome of PCC 6803.

### 4. Measurement of Chlorophyll and Carotenoid Contents

Initially, 200 μL of samples were collected and centrifuged at 10000 rpm for 5 minutes. Supernatant were removed from the solution and 200 μL of pure methanol was added to each sample and mixed properly using vortex. Then, samples kept in 4° C. for 20 minutes and centrifuged at 10000 for 1 minutes. The pigment concentrations were taken using optical density at 470 nm, 665 nm, 720 nm and calculated from Eq. (1) and Eq. (2).

$$Chla content ( µg / ml ) = 12.9447 ( A 665 - A 720 ) \qquad ( 1 )$$

$$Carotenoids ( µg / ml ) = \frac{[ 1000 ( A 470 - A 720 ) - 286 ( Chla [ µg / ml ] ) ]}{221} \qquad ( 2 )$$

### 5. Oxygen Evolution Study

A Hansatech Oxytherm+P (photosynthesis) system was used to measure the oxygen evolved by the cyanobacteria strains. This system contains a S1 Clark-type oxygen electrode disc, a magnetic stirrer, a vessel for samples, two high intensity LED lights surrounding the vessel, and a plunger to close the chamber (not to let environment oxygen inside the camber). S1 electrode disc is used to calculate the oxygen present in a chamber. The conditions used while performing this experiment were as follows: irradiation was fixed at 25 μmol photons, temperature inside the chamber was set at 30° C. throughout the experiment, stirrer speed was set at 80, 1 ml of samples were taken each time. Each time the 1 ml of samples were kept for 10 mins under 25 μE light.

### REFERENCES

1) Ermakova, M., Lopez-Calcagno, P. E., Raines, C. A., Furbank, R. T. and von Caemmerer, S., 2019. Overexpression of the Rieske FeS protein of the Cytochrome b 6 f complex increases C4 photosynthesis in Setaria viridis. Communications Biology, 2 (1), p.314.
2) Simkin, A. J., McAusland, L., Lawson, T. and Raines, C. A., 2017. Overexpression of the RieskeFeS protein increases electron transport rates and biomass yield. Plant physiology, 175 (1), pp. 134-145.

What is claimed:

1. A method of increasing biomanufacturing by a photosynthetic bacterium or a plant, the method comprising engineering the photosynthetic bacterium or plant to:
overexpress a cyt b₆f major protein, or
express a synthetic construct of a cyt b₆f major protein linked to a photosystem protein.

2. The method of claim 1, wherein the step of engineering the photosynthetic bacterium or plant to overexpress a cyt b₆f major protein comprises transforming the photosynthetic bacterium or a cell of the plant to express the cyt b₆f major protein using an overexpression promoter.

3. The method of claim 2, wherein the overexpression promoter is a tre promoter.

4. The method of claim 1, wherein the step of engineering the photosynthetic bacterium or plant to express the synthetic construct of the cyt b₆f major protein linked to a photosystem protein comprises:
transforming the photosynthetic bacterium or the cell of the plant to express a plasmid expressing a cyt b₆f major protein linked to a photosystem protein, wherein the operon expressing the photosystem protein in the genome of the transformed photosynthetic bacterium is replaced with a nucleotide sequence expressing the cyt b₆f major protein linked to the photosystem protein.

5. The method of claim 4, wherein the synthetic construct comprises a cyt b₆f major protein linked to a protein from Photosystem I.

6. The method of claim 5, wherein the plasmid comprises:
a first nucleotide sequence encoding petC:petA operon;
a second nucleotide sequence encoding a linker sequence;
a third nucleotide sequence encoding a psaA-psaB operon; and
the plasmid encodes a contiguous translation of any one of PetC, PetA, PsaA, PsaB, and/or a combination thereof.

7. The method of claim 6, wherein the petC:petA operon lacks the sequence encoding a stop codon of petA, and
wherein the stop codon is replaced by the second nucleotide sequence thus resulting in the expression of petA linked to psaA.

8. The method of claim 6, wherein the plasmid further comprises a fourth sequence encoding a petC promoter, wherein the petC promoter controls transcription of petC, petA, psaA, and psaB.

9. The method of claim 6, wherein the plasmid comprises a fifth nucleotide sequence and a sixth nucleotide sequence, and
wherein the fifth and the sixth nucleotide sequences are complementary to sequences flanking either and/or both of the 5′ and 3′ termini of the psaA-psaB operon of a cyanobacteria genome.

10. The method of claim 9, wherein the fifth nucleotide sequence and the sixth nucleotide sequences are each about 1 kb and are complementary to about a 1 kb sequence flanking either and/or both of the 5′ and 3′ termini of the psaA-psaB operon of the cyanobacteria genome.

11. The method of claim 4, wherein the synthetic construct comprises the cyt b₆f linked to a protein from Photosystem II.

12. The method of claim 4, wherein the synthetic construct comprises a linker sequence having an amino acid sequence set forth in SEQ ID NO. 35, wherein the linker sequence links the cyt b₆f major protein to the photosystem protein.

13. The method of claim 6, wherein the second nucleotide sequence comprises the sequence set forth in SEQ ID NO. 34 or the linker sequence comprises an amino acid sequence set forth in SEQ ID NO. 35.

14. The method of claim 1, wherein the engineered photosynthetic bacterium or plant exhibits increased photosynthetic efficiency compared to its native counterpart, and
wherein the increased photosynthetic efficiency results in increased cell growth and/or biomass production.

15. An engineered cyanobacterium overexpressing a cyt b₆f major protein, wherein gene expression of the cyt b₆f major protein is at least under the regulation of a tre promoter (P_{trc}).

16. The engineered cyanobacterium of claim 15, wherein the cyt b₆f major protein is PetD.

17. An engineered cyanobacterium expressing a synthetic construct of cyt b₆f major protein linked to a photosystem protein.

18. The engineered cyanobacterium of claim 17, wherein:
the synthetic construct comprises the cyt b₆f major protein linked to the protein from Photosystem I;
the synthetic construct is expressed via a modified psaA-psaB operon region; and
wherein
the modified psaA-psaB operon region comprises:
a first nucleotide sequence encoding petC:petA operon;
a second nucleotide sequence encoding a linker sequence; and
a third nucleotide sequence encoding a psaA-psaB operon.

19. The engineered cyanobacterium of claim 18, wherein modified psaA-psaB operon region further comprises a fourth sequence encoding a petC promoter, and
wherein the petC promoter controls transcription of petC, petA, psaA, and psaB.

20. The engineered cyanobacterium of claim 19, wherein the cyanobacterium is Synechocystis.
